(19) United States
(12) Patent Application Publication
(10) Pub. No.: US 2024/0192229 A1
(43) Pub. Date: Jun. 13, 2024

(54) METHODS AND MATERIALS FOR DETECTING MISFOLDED POLYPEPTIDES

(71) Applicant: Regents of the University of Minnesota, Minneapolis, MN (US)

(72) Inventors: Peter C. Christenson, Minneapolis, MN (US); Gage Rowden, Minneapolis, MN (US); Sang-Hyun Oh, Minneapolis, MN (US); Peter A. Larsen, Minneapolis, MN (US); Manci Li, Minneapolis, MN (US)

(21) Appl. No.: 18/286,682

(22) PCT Filed: Apr. 15, 2022
(86) PCT No.: PCT/US2022/025059

§ 371 Date: Oct. 12, 2023

Related U.S. Application Data
(60) Provisional application No. 63/277,999, filed on Nov. 10, 2021.
(60) Provisional application No. 63/176,114, filed on Apr. 16, 2021.

(51) Int. Cl. G01N 33/68; G01N 33/58

(52) U.S. Cl. CPC G01N 33/6896; G01N 33/587; G01N 2800/2821; G01N 2800/2828; G01N 2800/2835

(57) ABSTRACT

This document relates to methods and materials for detecting the presence or absence of misfolded polypeptides in a sample. For example, methods and materials for amplifying a sample (e.g., a biological sample or an environmental sample) such that misfolded polypeptides present in the sample can aggregate to form fibrils and/or globular polypeptide aggregates and contacting the amplified sample with a solution containing metal nanoparticles (e.g., gold nanoparticles) or one or more organic dyes (e.g., Congo Red) to detect the presence or absence of fibrils and/or globular polypeptide aggregates are provided. In some cases, methods and materials for determining if a mammal (e.g., a human) has a proteinopathy based, at least in part, in the presence or absence of misfolded polypeptides in a sample obtained from the mammal are provided.

### CROSS-REFERENCE TO RELATED APPLICATIONS

This application claims the benefit of U.S. Patent Application Ser. No. 63/176,114, filed on Apr. 16, 2021, and the benefit of U.S. Patent Application Ser. No. 63/277,999, filed on Nov. 10, 2021. The disclosure of the prior application is considered part of (and is incorporated by reference in) the disclosure of this application.

### TECHNICAL FIELD

This document relates to methods and materials for detecting the presence or absence of misfolded polypeptides in a sample. For example, a sample (e.g., a biological sample or an environmental sample) can be amplified as described herein such that misfolded polypeptides present in the sample can aggregate to form fibrils and/or globular polypeptide aggregates, and the amplified sample can be contacted with a solution containing nanoparticles (e.g., metal nanoparticles such as gold nanoparticles) as described herein to detect the presence or absence of fibrils and/or globular polypeptide aggregates. In another example, a sample (e.g., a biological sample or an environmental sample) can be amplified as described herein such that misfolded polypeptides present in the sample can aggregate to form fibrils and/or globular polypeptide aggregates, and the amplified sample can be contacted with a solution containing one or more organic dyes (e.g., Congo Red) as described herein to detect the presence or absence of fibrils and/or globular polypeptide aggregates. In some cases, methods and materials provided herein can be used to determine if a mammal (e.g., a human) has a proteinopathy based, at least in part, in the presence or absence of misfolded polypeptides in a sample obtained from the mammal.

### BACKGROUND INFORMATION

Prion diseases, collectively identified as transmissible spongiform encephalopathies (TSEs), are caused by an infectious misfolded prion proteins that spread throughout an infected animal, ultimately leading to advanced neurodegeneration and death (Prusiner et al., Proc. Nat. Acad. Sci. USA, 95:13363-13383 (1998); and Williams et al., J. Wildlife Dis., 16:89-98 (1980)). TSEs are known to infect a wide variety of mammals including cattle, sheep, camels, mink, cats, cervids, and humans (Collinge, Ann. Rev. Neurosci., 24:519-550 (2001)). Similarly, protein-misfolding diseases of humans, such as sporadic Alzheimer's Disease (AD), Parkinson's Disease (PD), Pick's disease, Lewy body dementia (LBD), and amyotrophic lateral sclerosis, are thought to originate from the misfolding and pathogenic accumulation of proteins (e.g., amyloid plaques and tau tangles) within the central nervous system (Lin et al., Nature, 443:787-795 (2006)).

### SUMMARY

This document provides methods and materials for detecting the presence or absence of misfolded polypeptides in a sample. For example, a sample (e.g., a biological sample or an environmental sample) can be amplified such that misfolded polypeptides present in the sample can aggregate to form fibrils and/or globular polypeptide aggregates, and the amplified sample can be contacted with a solution containing nanoparticles (e.g., metal nanoparticles such as gold nanoparticles) to detect the presence or absence of fibrils and/or globular polypeptide aggregates. In another example, a sample (e.g., a biological sample or an environmental sample) can be amplified such that misfolded polypeptides present in the sample can aggregate to form fibrils and/or globular polypeptide aggregates, and the amplified sample can be contacted with a solution containing one or more organic dyes (e.g., Congo Red) to detect the presence or absence of fibrils and/or globular polypeptide aggregates. In some cases, methods and materials provided herein can be used to determine if a mammal (e.g., a human) has a proteinopathy based, at least in part, in the presence or absence of misfolded polypeptides in a sample obtained from the mammal. For example, a sample obtained from a mammal can be amplified such that misfolded polypeptides present in the sample can aggregate to form fibrils, the amplified sample can be contacted with a solution containing nanoparticles (e.g., metal nanoparticles such as gold nanoparticles) to detect the presence or absence of fibrils, and the mammal can be classified as having a proteinopathy if the presence of fibrils is detected. For example, a sample obtained from a mammal can be amplified such that misfolded polypeptides present in the sample can aggregate to form globular polypeptide aggregates, the amplified sample can be contacted with a solution containing nanoparticles (e.g., metal nanoparticles such as gold nanoparticles) to detect the presence or absence of globular polypeptide aggregates, and the mammal can be classified as having a proteinopathy if the presence of globular polypeptide aggregates is detected. In another example, a sample obtained from a mammal can be amplified such that misfolded polypeptides present in the sample can aggregate to form fibrils, the amplified sample can be contacted with a solution containing one or more organic dyes (e.g., Congo Red) to detect the presence or absence of fibrils, and the mammal can be classified as having a proteinopathy if the presence of fibrils is detected. For example, a sample obtained from a mammal can be amplified such that misfolded polypeptides present in the sample can aggregate to form globular polypeptide aggregates, the amplified sample can be contacted with a solution containing one or more organic dyes (e.g., Congo Red) to detect the presence or absence of globular polypeptide aggregates, and the mammal can be classified as having a proteinopathy if the presence of globular polypeptide aggregates is detected.

As demonstrated herein, amplifying a sample suspected of containing misfolded polypeptides, and contacting the amplified sample with a solution of gold nanoparticles or Congo Red can be used to detect the presence or absence of the misfolded polypeptides. For example, samples containing prions from deer having chronic wasting disease (CWD) were amplified such that the prions within the samples formed fibrils, the amplified samples were contacted with a solution of gold nanoparticles, and the presence of the amplified prions was visually detected by the color of the solution and was detected by the peak light absorbance of the solution. In another example, samples containing misfolded α-synuclein polypeptides were amplified such that the misfolded α-synuclein polypeptides within the samples formed fibrils, the amplified samples were contacted with a solution of gold nanoparticles or Congo Red, and the presence of the amplified α-synuclein polypeptides was visually detected by the color of the solution and was detected by the peak light absorbance of the solution.

Having the ability to detect the presence of misfolded polypeptides in a sample as described herein (e.g., by amplifying the sample and contacting the amplified sample with a solution containing metal nanoparticles or one or more organic dyes such as Congo Red) provides a unique method to quickly and effectively detect the presence of misfolded polypeptides. For example, the methods described herein provide a protein-based diagnostic method that is relatively easy to use, requires inexpensive reagents and equipment, is highly sensitive and specific to the targeted protein, and can be performed in diverse settings (e.g., in field settings, small laboratories, etc.). In some cases, the methods described herein can be used to quickly and easily identify a mammal as having a proteinopathy.

In general, one aspect of this document features methods for detecting the presence or absence of misfolded polypeptides in a sample. The methods can include, or consist essentially of, (a) amplifying a sample under conditions where the misfolded polypeptides, when present, form fibrils; (b) contacting the sample with a solution containing metal nanoparticles; (c) detecting the fibrils in the solution containing the metal nanoparticles; (d) identifying the sample as having the presence of the misfolded polypeptides if the fibrils are detected; and (e) identifying the sample as lacking the misfolded polypeptides if the fibrils are not detected. The sample can be a biological sample. The biological sample can be obtained from a living mammal. The living mammal can be a human, a monkey, a camel, a horse, a mink, a cat, a cow, a sheep, a mouse, a rat, a hamster, a brocket, a chital, an elk, a fallow deer, a marsh deer, a mule deer, a muntjac, a moose, a pampas deer, a red deer, a reindeer, a roe deer, a sambar deer, a sika, a white-tailed deer, an antelope, or a goat. The biological sample can be lymph tissue, muscle tissue, tonsil tissue, skin tissue, brain tissue, brain-stem tissue, blood, cerebrospinal fluid, urine, feces, saliva, mucus, liver tissue, heart tissue, intestinal tissue, spleen tissue, or eye tissue. The biological can be obtained from a mammal post-mortem. The biological sample can be beef or venison. The sample can be an environmental sample. The environmental sample can be soil, water, dust, or a plant. The environmental sample can be obtained using a swab or a filter. The environmental sample can be obtained from a location selected from group consisting of a natural habitat, a waterway, a farm, a food processing facility, a water-treatment facility, and a hospital. When the environmental sample is obtained from a food processing facility, the food processing facility can process food intended for mammalian consumption. The environmental sample can be obtained from a hospital. The method can include, prior to the amplifying step, isolating polypeptides from the sample. The amplifying step can include shaking the sample or sonicating the sample. The metal nanoparticles can be gold nanoparticles. The detecting step can include visually detecting a color shift, where the color shift is indicative of the absence of the misfolded polypeptide. In some cases, the detecting step can include detecting light absorbance, where an absorbance of from about 510 nm to about 525 nm (e.g., about 521 nm) is indicative of the presence of the misfolded polypeptide, and where an absorbance of from about 530 nm to about 600 nm is indicative of the absence of the misfolded polypeptide. In some cases, the detecting step can include detecting light absorbance, where an absorbance of from about 510 nm to about 521 nm (e.g., about 517 nm) is indicative of the presence of the misfolded polypeptide, and where an absorbance of from about 525 nm to about 600 nm is indicative of the absence of the misfolded polypeptide. The misfolded polypeptide can be a prion protein (PrP) polypeptide, a tau polypeptide, an amyloid ß polypeptide, an α-synuclein polypeptide, or a TDP-43 polypeptide. The misfolded polypeptide can be associated with a proteinopathy. The proteinopathy can be chronic wasting disease (CWD), Cruzefeldt-Jakob Disease, transmissible mink encephalopathy, feline spongiform encephalopathy, ungulate spongiform encephalopathy, bovine-spongiform encephalapothy, camilid spongiform encephalopathy, pituitary pars intermedia dysfunction (PPID), Alzheimer's Disease (AD), Parkinson's Disease (PD), Pick's disease, Lewy body dementia (LBD), amyotrophic lateral sclerosis (ALS), multiple systems atrophies, progressive supranuclear palsies, corticobasal degenerations, or a chronic traumatic encephalopathy.

In another aspect, this document features methods for detecting the presence or absence of misfolded polypeptides in a sample. The methods can include, or consist essentially of, (a) amplifying a sample under conditions where the misfolded polypeptides, when present, form globular polypeptide aggregates; (b) contacting the sample with a solution containing metal nanoparticles; (c) detecting the globular polypeptide aggregates in the solution containing the metal nanoparticles; (d) identifying the sample as having the presence of the misfolded polypeptides if the globular polypeptide aggregates are detected; and (e) identifying the sample as lacking the misfolded polypeptides if the globular polypeptide aggregates are not detected. The sample can be a biological sample. The biological sample can be obtained from a living mammal. The living mammal can be a human, a monkey, a camel, a horse, a mink, a cat, a cow, a sheep, a mouse, a rat, a hamster, a brocket, a chital, an elk, a fallow deer, a marsh deer, a mule deer, a muntjac, a moose, a pampas deer, a red deer, a reindeer, a roe deer, a sambar deer, a sika, a white-tailed deer, an antelope, or a goat. The biological sample can be lymph tissue, muscle tissue, tonsil tissue, skin tissue, brain tissue, brain-stem tissue, blood, cerebrospinal fluid, urine, feces, saliva, mucus, liver tissue, heart tissue, intestinal tissue, spleen tissue, or eye tissue. The biological can be obtained from a mammal post-mortem. The biological sample can be beef or venison. The sample can be an environmental sample. The environmental sample can be soil, water, dust, or a plant. The environmental sample can be obtained using a swab or a filter. The environmental sample can be obtained from a location selected from group consisting of a natural habitat, a waterway, a farm, a food processing facility, a water-treatment facility, and a hospital. When the environmental sample is obtained from a food processing facility, the food processing facility can process food intended for mammalian consumption. The environmental sample can be obtained from a hospital. The method can include, prior to the amplifying step, isolating polypeptides from the sample. The amplifying step can include shaking the sample or sonicating the sample. The metal nanoparticles can be gold nanoparticles. The detecting step can include visually detecting a color shift, where the color shift is indicative of the absence of the misfolded polypeptide. In some cases, the detecting step can include detecting light absorbance, where an absorbance of from about 510 nm to about 525 nm (e.g., about 521 nm) is indicative of the presence of the misfolded polypeptide, and where an absorbance of from about 530 nm to about 600 nm is indicative of the absence of the misfolded polypeptide. In some cases, the detecting step can include detecting light absorbance, where an absorbance of from about 510 nm to about 521 nm (e.g., about 517 nm) is indicative of the presence of the misfolded polypeptide, and where an absorbance of from about 525 nm to about 600 nm is indicative of the absence of the misfolded polypeptide. The misfolded polypeptide can be a PrP polypeptide, a tau polypeptide, an amyloid ß polypeptide, an α-synuclein polypeptide, or a TDP-43 polypeptide. The misfolded polypeptide can be associated with a proteinopathy. The proteinopathy can be CWD, Cruzefeldt-Jakob Disease, transmissible mink encephalopathy, feline spongiform encephalopathy, ungulate spongiform encephalopathy, bovine-spongiform encephalapothy, camilid spongiform encephalopathy, PPID, AD, PD, Pick's disease, LBD, ALS, multiple systems atrophies, progressive supranuclear palsies, corticobasal degenerations, or a chronic traumatic encephalopathy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. A schematic representation of an exemplary Minnesota Quaking-Induced Conversion (MN-QuIC) assay.

FIGS. 2A-2C. Gold nanoparticles reflect prion misfolding. FIG. 2A: EP-QuIC results showing relative Thioflavin T (ThT) fluorescence (RFU) of CWD+ and CWD-samples, including positive and negative controls. FIG. 2B: MN-QuIC results reveal a spectral shift in absorbance maxima depending on whether there is prion fibrillation (as shown in FIG. 2A). FIG. 2C: For each analysis, the results were pooled (including controls) depending on their independently determined CWD status and a non-parametric u-test was performed. CWD positive vs. negative samples were determined to be statistically different (P<0.0001) for both EP-QuIC and MN-QuIC analyses.

FIGS. 3A-3C. Gold nanoparticles (AuNPs) can distinguish normal vs misfolded prions. Dynamic light scattering (DLS) data showing the distributions of the size of AuNPs for samples containing no protein present in AuNPs (FIG. 3A), samples containing recombinant prion proteins in AuNPs (FIG. 3B), and samples containing misfolded recombinant prion fibrils in AuNPs (FIG. 3C). As nanoparticles aggregate they form bigger collections of particles that are detected by the DLS. Data are shown as a percentage of the total volume of AuNPs present that each size contains. There is little difference between the no protein and positive fibril sample.

FIG. 4A-C. Direct comparison of RT-QuIC and MN-QuIC using the same sample set. FIG. 4A: Rate of amyloid formation, reflecting the amount of prion fibrils initially present in the sample, from RT-QuIC was plotted for 5 CWD⁺ and 5 CWD samples. FIG. 4B: Wavelength for absorbance maximum from MN-QuIC for samples described in FIG. 4A. FIG. 4C: Color change reflecting AuNP absorbance peak shift shown in FIG. 4B was captured by camera. Samples identified by + indicate positive controls and − indicate negative controls.

FIG. 5. Images of the color change observed in samples containing misfolded polypeptides. Each row shows the same tubes but the pictures were taken using different camera filters.

FIG. 6. Absorbance spectrum shift in CWD negative versus positive samples.

FIG. 7. Peak absorbance wavelength in CWD negative versus positive samples.

FIG. 8. Overview of the MN-QuIC assay. Misfolded chronic wasting disease (CWD) prion seeds originating from biological samples of cervids are added to rPrP solutions. These solutions are then shaken and incubated for approximately 24 hours. If present, PrPCWD induces conformational changes of the rPrP. Resulting products are diluted and added to an AuNP solution. CWD positive samples result in a red solution (peak absorbance wavelength ˜516 nm) while CWD negative solutions are purple (peak absorbance wavelength ˜560 nm).

FIGS. 9A-9F. FIG. 9A: The relative fluorescence units of post QuIC solutions containing misfolded protein seeds and solutions without misfolded protein seeds. FIG. 9B: The absorbance spectrum of AuNP solutions spiked with misfolded rHaPrP from seeded reactions and non-misfolded/native rHaPrP from reactions without seed. FIG. 9C: Average particle sizes in AuNP solutions containing no protein, misfolded rHaPrP, and native rHaPrP observed by dynamic light scattering reading (DLS). FIG. 9D: DLS readings of AuNP solution with no protein added. Reported as percent of total volume of AuNPs present. FIG. 9E: DLS readings of AuNP solution with misfolded rHaPrP. FIG. 9F: DLS readings of AuNP solution with native rHaPrP. *, p-value <0.05, error bars show standard deviation.

FIGS. 10A-10C. FIG. 10A: RT-QuIC data for the rate of amyloid formation for negative and positive medial retropharyngeal lymph node tissue samples from wild white-tailed deer. Sample identification number on horizontal axis. FIG. 10B: Photo of MN-QuIC tubes showing the color difference for the same set of tissue samples used in panel A. C) MN-QuIC peak absorbance wavelength of the same set of solutions used in panel B. *, p-value <0.05, error bars show standard deviation.

FIG. 11. Wavelength of peak absorbance of AuNPs in different pH buffers containing native CWD negative rHaPrP (square), PrPCWD positive (circle), and blank/no protein (triangle) solutions.

FIGS. 12A and 12B. FIG. 12A: RT-QuIC data for tonsil samples used in study.

FIG. 12B: Number of red wells out of the 8 replicates for each animal tested. ** p<. 01, error bars show standard deviation.

FIG. 13. Prion seeding activity as shown by time (in hours) to Thioflavin-T fluorescence threshold via RT-QuIC reactions for various lymph tissues.

FIG. 14. A graph showing the ratio of the absorbance at 517 nm divided by the absorbance at 580 nm (517/580 ratio) of 10 nm AuNPs or 15 nm AuNPs in CWD positive samples or CWD negative samples.

FIG. 15. Wavelength of peak absorbance of AuNPs in CWD positive samples or CWD negative samples shaken for 20 hours.

FIG. 16. Wavelength of peak absorbance of AuNPs in recombinant prion substrate seeded with α-synuclein positive samples or α-synuclein negative samples.

FIGS. 17A and 17B. Congo Red can distinguish normal vs misfolded α-synuclein. FIG. 17A: Color change of Congo Red observed in α-synuclein positive (misfolded) samples and α-synuclein negative (non-misfolded) samples. FIG. 17B: Wavelength of peak absorbance of different ratios of Congo Red to polypeptides in α-synuclein positive samples or α-synuclein negative samples.

### DETAILED DESCRIPTION

This document provides methods and materials for detecting the presence or absence of misfolded polypeptides, polypeptide fibrils, and/or polypeptide aggregates (e.g., globular polypeptide aggregates) in a sample. For example, a sample (e.g., a biological sample or an environmental sample) can be amplified such that misfolded polypeptides present in the sample form fibrils and/or globular polypeptide aggregates, and the amplified sample can be contacted with a solution containing nanoparticles (e.g., metal nanoparticles such as gold nanoparticles) to detect the presence or absence of fibrils and/or polypeptide aggregates (e.g., globular polypeptide aggregates). Nanoparticles (e.g., metal nanoparticles such as gold nanoparticles) used in the methods described herein have unique optical properties. For example, when light interacts with the surface electrons of a metal nanoparticle, it causes the surface electrons to oscillate. At certain wavelengths, oscillations can be in phase and can cause plasmonic resonance, and at the wavelength of plasmonic resonance, metal nanoparticles exhibit very large absorption. When metal nanoparticles within a solution aggregate, the absorption of the solutions shifts. Accordingly, solutions containing metal nanoparticles (e.g., gold nanoparticles) can be used to detect the presence or absence of misfolded polypeptides. For example, a sample suspected of containing misfolded polypeptides can be amplified such that misfolded polypeptides, when present, can aggregate to form fibrils (e.g., aggregates of two or more misfolded polypeptides), and the amplified sample can be contacted with a solution containing metal nanoparticles (e.g., gold nanoparticles) to detect the presence or absence of fibrils. When fibrils are present in a solution containing metal nanoparticles (e.g., gold nanoparticles), the metal nanoparticles lack any change in absorption. For example, when fibrils are present in a solution containing metal nanoparticles (e.g., gold nanoparticles), the solution can appear red based on its absorption spectrum. When fibrils are not present in a solution containing metal nanoparticles (e.g., gold nanoparticles), the metal nanoparticles aggregate thereby causing a detectable change in absorption. For example, when fibrils are not present in a solution containing metal nanoparticles (e.g., gold nanoparticles), the solution can appear blue based on its absorption spectrum.

In some cases, a sample (e.g., a biological sample or an environmental sample) can be amplified such that misfolded polypeptides present in the sample form fibrils and/or globular polypeptide aggregates, and the amplified sample can be contacted with a solution containing one or more organic dyes (e.g., Congo Red) to detect the presence or absence of fibrils and/or polypeptide aggregates (e.g., globular polypeptide aggregates). Organic dyes (e.g., Congo Red) used in the methods described herein can have colloidal particles that provide unique optical properties. For example, when light interacts with a solution containing an organic dye in a suspension, it causes misfolded proteins can cause the organic dye to turn blue. When misfolded proteins within a solution aggregate, the absorption of the organic dye solution shifts. Accordingly, solutions containing one or more organic dyes (e.g., Congo Red) can be used to detect the presence or absence of misfolded polypeptides. For example, a sample suspected of containing misfolded polypeptides can be amplified such that misfolded polypeptides, when present, can aggregate to form fibrils (e.g., aggregates of two or more misfolded polypeptides), and the amplified sample can be contacted with a solution containing one or more organic dyes (e.g., Congo Red) to detect the presence or absence of fibrils. When fibrils are present in a solution containing one or more organic dyes (e.g., Congo Red), the organic dye(s) cause a detectable change in absorption. For example, when fibrils are present in a solution containing one or more organic dyes (e.g., Congo Red), the solution can appear blue (e.g., as compared to a solution that lacks fibrils) based on its absorption spectrum. When fibrils are not present in a solution containing one or more organic dyes (e.g., Congo Red), the organic dye(s) lack any change in absorption. For example, when fibrils are not present in a solution containing one or more organic dyes (e.g., Congo Red), the solution can appear red (e.g., as compared to a solution that contains fibrils) based on its absorption spectrum.

In some cases, the methods described herein (e.g., the methods for detecting the presence or absence of a misfolded polypeptide) can be used to detect the presence or absence of one or more aggregates of misfolded polypeptides. For example, the methods described herein can be used to detect the presence or absence of amyloid plaques. For example, the methods described herein can be used to detect the presence or absence of tau tangles.

In some cases, the methods described herein (e.g., the methods for detecting the presence or absence of a misfolded polypeptide) can be used to determine if a mammal (e.g., a human) has a proteinopathy based, at least in part, in the presence or absence of misfolded polypeptides in a sample obtained from the mammal. For example, a sample obtained from a mammal can be amplified such that misfolded polypeptides present in the sample can aggregate to form fibrils, the amplified sample can be contacted with a solution containing nanoparticles (e.g., metal nanoparticles such as gold nanoparticles) to detect the presence or absence of fibrils, and the mammal can be classified as having a proteinopathy if the presence of fibrils is detected. For example, a sample obtained from a mammal can be amplified such that misfolded polypeptides present in the sample can aggregate to form fibrils, the amplified sample can be contacted with a solution containing nanoparticles (e.g., metal nanoparticles such as gold nanoparticles) to detect the presence or absence of fibrils, and the mammal can be classified as not having a proteinopathy if the absence of fibrils is detected. In another example, a sample obtained from a mammal can be amplified such that misfolded polypeptides present in the sample can aggregate to form fibrils, the amplified sample can be contacted with a solution containing one or more organic dyes (e.g., Congo Red) to detect the presence or absence of fibrils, and the mammal can be classified as having a proteinopathy if the presence of fibrils is detected. For example, a sample obtained from a mammal can be amplified such that misfolded polypeptides present in the sample can aggregate to form fibrils, the amplified sample can be contacted with a solution containing one or more organic dyes (e.g., Congo Red) to detect the presence or absence of fibrils, and the mammal can be classified as not having a proteinopathy if the absence of fibrils is detected. In some cases, this document provides methods and materials for treating a mammal (e.g., a human) identified as having a proteinopathy as described herein (e.g., based, at least in part, on the presence of fibrils formed from misfolded polypeptides).

Any appropriate method can be used to amplify a sample (e.g., a sample suspected of containing misfolded polypeptides). In some cases, a sample can be amplified by shaking. A sample can be shaken for any appropriate amount of time. For example, a sample can be shaken for from about 3 hours to about 40 hours (e.g., from about 3 hours to about 36 hours, from about 3 hours to about 32 hours, from about 3 hours to about 24 hours, from about 3 hours to about 18 hours, from about 3 hours to about 15 hours, from about 3 hours to about 12 hours, from about 3 hours to about 8 hours, from about 5 hours to about 40 hours, from about 8 hours to about 40 hours, from about 12 hours to about 40 hours, from about 18 hours to about 40 hours, from about 22 hours to about 40 hours, from about 24 hours to about 40 hours, from about 28 hours to about 40 hours, from about 32 hours to about 40 hours, from about 6 hours to about 32 hours, from about 12 hours to about 28 hours, from about 15 hours to about 22 hours, from about 6 hours to about 18 hours, from about 12 hours to about 24 hours, from about 15 hours to about 28 hours, or from about 18 hours to about 32 hours). In some cases, a sample can be shaken for about 24 hours. A sample can be shaken at any appropriate speed. For example, a sample can be shaken at from about 200 RPM to about 1000 RPM (e.g., from about 200 RPM to about 800 RPM, from about 200 RPM to about 600 RPM, from about 200 RPM to about 400 RPM, from about 400 RPM to about 1000 RPM, from about 600 RPM to about 1000 RPM, from about 800 RPM to about 1000 RPM, from about 400 RPM to about 800 RPM, from about 500 RPM to about 700 RPM, from about 200 RPM to about 400 RPM, from about 400 RPM to about 600 RPM, or from about 600 RPM to about 800 RPM). In some cases, a sample can be shaken at from about 600 RPM to about 700 RPM. A sample can be shaken at any appropriate temperature. For example, a sample can be shaken at from about 30° C. to about 65° C. (e.g., from about 30° C. to about 60° C., from about 30° C. to about 55° C., from about 30° C. to about 50° C., from about 30° C. to about 45° C., from about 30° C. to about 40° C., from about 30° C. to about 35° C., from about 35° C. to about 65° C., from about 40° C. to about 65° C., from about 45° C. to about 65° C., from about 50° C. to about 65° C., from about 55° C. to about 65° C., from about 33° C. to about 60° C., from about 40° C. to about 55° C., from about 40° C. to about 50° C., or from about 50° C. to about 60° C.). In some cases, a sample can be shaken at about 42° C.

In some cases, a sample can be amplified by sonication. A sample can be sonicated for any appropriate amount of time. For example, a sample can be sonicated for from about 5 seconds to about 30 seconds (e.g., from about 5 seconds to about 25 seconds, from about 5 seconds to about 20 seconds, from about 5 seconds to about 15 seconds, from about 5 seconds to about 10 seconds, from about 10 seconds to about 30 seconds, from about 15 seconds to about 30 seconds, from about 20 seconds to about 30 seconds, from about 25 seconds to about 30 seconds, from about 10 seconds to about 25 seconds, from about 15 seconds to about 20 seconds, from about 10 seconds to about 15 seconds, or from about 20 seconds to about 25 seconds). A sonication step can be performed any number of times. For example, a sample can be sonicated from about 2 times to about 10 times (e.g., from about 2 times to about 8 times, from about 2 times to about 6 times, from about 2 times to about 4 times, from about 4 times to about 10 times, from about 6 times to about 10 times, from about 8 times to about 10 times, from about 4 times to about 8 times, from about 4 times to about 6 times, or from about 6 times to about 8 times). A sonication step can be performed any number of times. A sonication step can be performed at any temperature. For example, a sample can be sonicated at from about 30° C. to about 65° C.

In some cases, an amplified sample can be contacted with a solution containing nanoparticles (e.g., metal nanoparticles such as gold nanoparticles) or one or more organic dyes (e.g., Congo Red) immediately after amplification. For example, an amplified sample can be contacted with a solution containing nanoparticles (e.g., metal nanoparticles such as gold nanoparticles) or one or more organic dyes (e.g., Congo Red) from about 1 minute to about 60 minutes (e.g., from about 1 minute to about 45 minutes, from about 1 minute to about 30 minutes, from about 1 minute to about 20 minutes, from about 1 minute to about 10 minutes, from about 10 minutes to about 60 minutes, from about 20 minutes to about 60 minutes, from about 30 minutes to about 60 minutes, from about 45 minutes to about 60 minutes, from about 10 minutes to about 45 minutes, from about 20 minutes to about 30 minutes, from about 10 minutes to about 30 minutes, or from about 30 minutes to about 45 minutes) after amplification.

In some cases, an amplified sample can be stored (e.g., at 4° C. for an indefinite amount of time prior to being contacted with a solution containing nanoparticles (e.g., metal nanoparticles such as gold nanoparticles) or one or more organic dyes (e.g., Congo Red).

An amplified sample can be contacted with any appropriate solution containing nanoparticles (e.g., metal nanoparticles such as gold nanoparticles) or one or more organic dyes (e.g., Congo Red). In some cases, a solution can include disodium phosphate (Na₂HPO₄). For example, a solution can include from about 5 mM to about 15 mM disodium phosphate (e.g., from about 5 mM to about 12 mM, from about 5 mM to about 10 mM, from about 5 mM to about 8 mM, from about 7 mM to about 15 mM, from about 10 mM to about 15 mM, from about 12 mM to about 15 mM, from about 8 mM to about 12 mM, from about 7 mM to about 10 mM, from about 10 mM to about 12 mM, or about 10 mM disodium phosphate). In some cases, a solution can include potassium chloride (KCl). For example, a solution can include from about 1 mM to about 4 mM potassium chloride (e.g., from about 1 mM to about 3 mM, from about 1 mM to about 2 mM, from about 2 mM to about 4 mM, from about 3 mM to about 4 mM, from about 2 mM to about 3 mM, or about 2.7 mM potassium chloride). In some cases, a solution can include monopotassium phosphate (KH₂PO₄). For example, a solution can include from about 1 mM to about 3 mM monopotassium phosphate (e.g., from about 1 mM to about 2 mM, from about 2 mM to about 3 mM, or about 1.8 mM monopotassium phosphate). In some cases, a solution can have a pH of from about 5 to about 9 (e.g., from about 5 to about 8, from about 5 to about 7, from about 5 to about 6, from about 6 to about 9, from about 7 to about 9, from about 8 to about 9, from about 6 to about 8, from about 6 to about 7, from about 7 to about 8, or a pH of about 7.41).

An amplified sample can be contacted with a solution containing any appropriate nanoparticles. In some cases, a nanoparticle can be a metal nanoparticle. A metal nanoparticle can be made from any appropriate metal. Examples of metals that can be used to make a metal nanoparticle include, without limitation, gold, silver, copper, platinum, iron, and alloys thereof. In some cases, a metal nanoparticle can be a gold nanoparticle. In some cases, a nanoparticle can be a quantum dot. Examples of quantum dots that can be used that can be used as a nanopoarticle in the methods described herein include, without limitation, CdS, CdSe, CdTe, ZnS, ZnSe, PbS, and InP. A nanoparticle can be any appropriate size (e.g., can have any appropriate longest dimension such as a diameter). In some cases, a nanoparticle can have a diameter of from about 1 nm to about 100 nm (e.g., from about 1 nm to about 80 nm, from about 1 nm to about 60 nm, from about 1 nm to about 40 nm, from about 1 nm to about 20 nm, from about 20 nm to about 100 nm, from about 40 nm to about 100 nm, from about 60 nm to about 100 nm, from about 80 nm to about 100 nm, from about 20 nm to about 80 nm, from about 40 nm to about 60 nm, from about 20 nm to about 40 nm, from about 30 nm to about 50 nm, from about 40 nm to about 60 nm, or from about 5 nm to about 70 nm). A nanoparticle can be any shape (e.g., a sphere, a rod, a nanowire, a shell, a cube, and a star).

In some cases, at least one nanoparticle (e.g., at least one metal nanoparticle) in a solution containing metal nanoparticles can be conjugated to another molecule. Examples of molecules that a nanoparticle in a solution containing nanoparticles can be conjugated to include, without limitation, a nanoparticle (e.g., a different nanoparticle such as a nanoparticle functionalized with citrate, cetyltrimethylammonium bromide, carboxylic Acid, poly(allylamine) hydrochloride, polyvinylpyrrolidone, poly(acrylic acid), polyethylene glycol, and/or polyethylenimine), nanobodies, biotinylated biomolecules, ligands and polypeptides such as antibodies.

An amplified sample can be contacted with a solution containing any appropriate organic dye(s). In some cases, an organic dye can be an azo dye. Examples of organic dyes that can be used as described herein include, without limitation, Congo Red, Nile Red, acridine orange, Trypan Blue, Evans Blue, Sirius Red f3b, primuline, X-34, 1,4-Bis(3-carboxy-4-hydroxyphenylethenyl)benzene, (trans,trans)-1-bromo-2,5-bis-(3-hydroxycarbonyl-4-hydroxy)styrylbenzene (BSB); BF-168, and (6-2-Fluoroethoxy)-2-[2-(4-methylaminophenil)ethenyl]benzoxazole. In some case, an organic dye that can be used as described herein can be as described elsewhere (see, e.g., Mishra et al., Mol. BioSyst., 7:1232-1240 (2011)).

In some cases, when an amplified sample is contacted with a solution containing nanoparticles (e.g., metal nanoparticles such as gold nanoparticles) or one or more organic dyes (e.g., Congo Red), the amplified sample and the solution containing nanoparticles (e.g., metal nanoparticles such as gold nanoparticles) or one or more organic dyes (e.g., Congo Red) can be incubated together (e.g., can be maintained in contact for an extended period of time) prior to detecting the presence or absence of fibrils. For example, an amplified sample and a solution containing nanoparticles (e.g., metal nanoparticles such as gold nanoparticles) or one or more organic dyes (e.g., Congo Red) can be incubated together for from about 2 minutes to about 60 minutes (e.g., from about 2 minutes to about 50 minutes, from about 2 minutes to about 40 minutes, from about 2 minutes to about 30 minutes, from about 2 minutes to about 20 minutes, from about 2 minutes to about 10 minutes, from about 2 minutes to about 5 minutes, from about 5 minutes to about 60 minutes, from about 10 minutes to about 60 minutes, from about 20 minutes to about 60 minutes, from about 30 minutes to about 60 minutes, from about 40 minutes to about 60 minutes, from about 50 minutes to about 60 minutes, from about 10 minutes to about 50 minutes, from about 20 minutes to about 40 minutes, from about 20 minutes to about 30 minutes, from about 30 minutes to about 40 minutes, or about 30 minutes).

Any appropriate method can be used to detect the presence or absence of fibrils and/or polypeptide aggregates (e.g., fibrils and/or polypeptide aggregates formed from misfolded polypeptides). In some cases, the presence or absence of fibrils in a solution containing nanoparticles (e.g., metal nanoparticles such as gold nanoparticles) or one or more organic dyes (e.g., Congo Red) that was contacted with an amplified sample can be detecting (e.g., visually detected) using color. For example, a solution containing nanoparticles (e.g., metal nanoparticles such as gold nanoparticles) that was contacted with an amplified sample containing fibrils can appear red (e.g., can appear red to the naked eye). For example, a solution containing nanoparticles (e.g., metal nanoparticles such as gold nanoparticles) that was contacted with an amplified sample lacking fibrils can appear blue (e.g., can appear blue to the naked eye). In some cases, the presence or absence of fibrils in a solution containing nanoparticles (e.g., metal nanoparticles such as gold nanoparticles) that was contacted with an amplified sample can be detecting using absorbance (e.g., absorbance of light such as visible light or near-infrared light). For example, a solution containing nanoparticles (e.g., metal nanoparticles such as gold nanoparticles) that was contacted with an amplified sample containing fibrils can have a wavelength of from about 510 nm to about 525 nm (e.g., about 516 nm or about 521 nm). In another example, a solution containing nanoparticles (e.g., metal nanoparticles such as gold nanoparticles) that was contacted with an amplified sample containing fibrils can have a wavelength of from about 510 nm to about 521 nm (e.g., about 516 nm or about 517 nm). For example, a solution containing nanoparticles (e.g., metal nanoparticles such as gold nanoparticles) that was contacted with an amplified sample lacking fibrils can have a wavelength of from about 530 nm to about 600 nm (e.g., from about 530 nm to about 575 nm, from about 530 nm to about 550 nm, from about 550 nm to about 600 nm, from about 575 nm to about 600 nm, or from about 550 nm to about 575 nm). In another example, a solution containing nanoparticles (e.g., metal nanoparticles such as gold nanoparticles) that was contacted with an amplified sample lacking fibrils can have a wavelength of from about 525 nm to about 600 nm (e.g., from about 530 nm to about 575 nm, from about 530 nm to about 550 nm, from about 550 nm to about 600 nm, from about 575 nm to about 600 nm, or from about 550 nm to about 575 nm).

For example, a solution containing one or more organic dyes (e.g., Congo Red) that was contacted with an amplified sample containing fibrils can appear blue (e.g., can appear blue to the naked eye). For example, a solution containing one or more organic dyes (e.g., Congo Red) that was contacted with an amplified sample lacking fibrils can appear red (e.g., can appear red to the naked eye). In some cases, the presence or absence of fibrils in a solution containing one or more organic dyes (e.g., Congo Red) that was contacted with an amplified sample can be detecting using absorbance (e.g., absorbance of light such as visible light or near-infrared light). For example, a solution containing one or more organic dyes (e.g., Congo Red) that was contacted with an amplified sample containing fibrils can have a wavelength of from about 494 nm to about 550 nm (e.g., from about 494 nm to about 525, from about 505 nm to about 550 nm, from about 515 nm to about 535 nm, from about 500 nm to about 550 nm, from about 525 nm to about 550 nm). For example, a solution containing one or more organic dyes (e.g., Congo Red) that was contacted with an amplified sample lacking fibrils can have a wavelength of from about 450 nm to about 493 nm (e.g., about 490 nm).

In some cases, the methods provided herein are not antibody-based methods. For example, the methods provided herein can be performed in the absence of antibody-based techniques.

In some cases, the methods provided herein can be performed in the absence of any stimulus. For example, the methods provided herein can be performed in the absence of electrochemical stimulus.

In some cases, the methods provided herein can be performed in the absence of any sensor. For example, the methods provided herein can be performed in the absence of any colorimetric sensor. For example, the methods provided herein can be performed in the absence of any electrochemical sensor.

The methods described herein (e.g., the methods for detecting the presence or absence of a misfolded polypeptide) can be used to detect the presence or absence of any misfolded polypeptide. In some cases, a misfolded polypeptide can be associated with a disease. Examples of polypeptides that can be misfolded, and where the misfolded polypeptide can be detected as described herein include, without limitation, prion protein (PrP) polypeptides, tau polypeptides, amyloid ß polypeptides, α-synuclein polypeptides, and TDP-43 polypeptides.

The methods described herein (e.g., the methods for detecting the presence or absence of a misfolded polypeptide) can be used to detect the presence or absence of a misfolded polypeptide associated with any proteinopathy. As used herein, a proteinopathy is any disease associated with misfolding and, optionally, aggregation of one or more of the misfolded polypeptides. In some cases, a proteinopathy can be a transmissible spongiform encephalopathy (TES). In some cases, a proteinopathy can be a protein-misfolding disease (PMD). In some cases, a proteinopathy can be a tauopathy. In some cases, a proteinopathy can be an α-synucleinopathy. Examples of proteinopathies associated polypeptides that can be misfolded, and where the misfolded polypeptide can be detected as described herein include, without limitation, chronic wasting disease (CWD), Cruzefeldt-Jakob Disease, transmissible mink encephalopathy, feline spongiform encephalopathy, ungulate spongiform encephalopathy, bovine-spongiform encephalapothy, camilid spongiform encephalopathy, pituitary pars intermedia dysfunction (PPID), Alzheimer's Disease (AD), Parkinson's Disease (PD), Pick's disease, Lewy body dementia (LBD), amyotrophic lateral sclerosis (ALS), multiple systems atrophies, progressive supranuclear palsies, corticobasal degenerations, and chronic traumatic encephalopathies.

The methods described herein (e.g., the methods for detecting the presence or absence of a misfolded polypeptide) can be used to detect the presence or absence of misfolded polypeptides in any appropriate sample. In some cases, a sample can be a biological sample (e.g., a sample obtained from a mammal). In some cases, a sample can be an environmental sample. A sample can be a fresh sample or a fixed sample (e.g., a formaldehyde-fixed sample or a formalin-fixed sample). In some cases, a sample can be a processed sample. For example, a processed sample can be homogenized. For example, a processed sample can be diluted (e.g., can be diluted in a buffer such as phosphate buffered saline (PBS)). In some cases, one or more biological molecules (e.g., polypeptides) can be isolated from a sample. For example, polypeptides can be isolated from a sample and can be enriched or concentrated prior to being amplified as described herein.

When the methods described herein (e.g., the methods for detecting the presence or absence of a misfolded polypeptide) are used to detect the presence or absence of misfolded polypeptides in a biological sample, the biological sample can be obtained from any appropriate mammal. In some cases, a sample can be obtained from a living mammal. In some cases, a sample can be obtained from a mammal post-mortem sample. For example, a post-mortem sample can be a mammalian tissue or byproduct intended for consumption by another mammal (e.g., a human) such as beef or venison. In some cases, a mammal can be a cervid (e.g., can be a member of the (′ervidae family). Examples of mammals that a sample can be obtained from and where the sample can be assessed for the presence or absence or misfolded polypeptides as described herein (e.g., by amplifying the sample and contacting the amplified sample with a solution containing nanoparticles (e.g., metal nanoparticles such as gold nanoparticles) or one or more organic dyes (e.g., Congo Red)) include, without limitation, humans, non-human primates (e.g., monkeys), camels, horses, mink, cats, cows, sheep, mice, rats, hamsters, brocket, chital, elk, fallow deer, marsh deer, mule deer, muntjac, moose, pampas deer, red deer, reindeer, roe deer, sambar deer, sika, white-tailed deer, antelope, and goats.

When the methods described herein (e.g., the methods for detecting the presence or absence of a misfolded polypeptide) are used to detect the presence or absence of misfolded polypeptides in a biological sample, the biological sample can be any type of biological sample. Examples of biological samples that can be assessed for the presence or absence or misfolded polypeptides as described herein (e.g., by amplifying the sample and contacting the amplified sample with a solution containing nanoparticles (e.g., metal nanoparticles such as gold nanoparticles) or one or more organic dyes (e.g., Congo Red)) include, without limitation, lymph tissue, muscle tissue, tonsil tissue, skin tissue, brain tissue, brain-stem tissue, blood, cerebrospinal fluid, urine, feces, saliva, mucus, liver tissue, heart tissue, intestinal tissue, spleen tissue, and eye tissue.

When the methods described herein (e.g., the methods for detecting the presence or absence of a misfolded polypeptide) are used to detect the presence or absence of misfolded polypeptides in an environmental sample, the environmental sample can be obtained from any appropriate source. Examples of sources that a sample can be obtained from and where the sample can be assessed for the presence or absence of misfolded polypeptides as described herein (e.g., by amplifying the sample and contacting the amplified sample with a solution containing nanoparticles (e.g., metal nanoparticles such as gold nanoparticles) or one or more organic dyes (e.g., Congo Red)) include, without limitation, soil, water, dust, and plants.

When the methods described herein (e.g., the methods for detecting the presence or absence of a misfolded polypeptide) are used to detect the presence or absence of misfolded polypeptides in an environmental sample, the environmental sample can be obtained by any appropriate method. Examples of methods that can be used to obtain an environmental sample that can be assessed for the presence or absence of misfolded polypeptides as described herein (e.g., by amplifying the sample and contacting the amplified sample with a solution containing nanoparticles (e.g., metal nanoparticles such as gold nanoparticles) or one or more organic dyes (e.g., Congo Red)) include, without limitation, swabs and filters (e.g., air filtration system filters).

When the methods described herein (e.g., the methods for detecting the presence or absence of a misfolded polypeptide) are used to detect the presence or absence of misfolded polypeptides in an environmental sample, the environmental sample can be obtained from any environmental setting. Examples of environmental settings that an environmental sample that can be assessed for the presence or absence of misfolded polypeptides as described herein (e.g., by amplifying the sample and contacting the amplified sample with a solution containing nanoparticles (e.g., metal nanoparticles such as gold nanoparticles) or one or more organic dyes (e.g., Congo Red)) can be obtained from include, without limitation, natural habitats, waterways, farms, food processing facilities (e.g., meat processing facilities), water-treatment facilities, and hospitals (e.g., human hospitals and veterinary hospitals). When an environmental sample is obtained from a food processing facility, the food processing facility can process food intended for mammalian (e.g., human) consumption. For example, an environmental sample can be obtained from a food processing facility that processes agricultural commodities (e.g., alfalfa, corn, beets, soybeans, oats, grasses, potatoes, straw, and related byproducts) for mammalian consumption. When an environmental sample is obtained from a hospital, the sample can be obtained from any surface (e.g., a stainless steel surface) in frequent contact with patients and/or biological fluids (e.g., blood, urine, and feces). For example, an environmental sample obtained from a hospital can be obtained from surgical tools, examination surfaces, and countertops.

In some cases, the presence or absence of misfolded polypeptides in a sample can be confirmed using one or more techniques traditionally used to detect the presence of the presence or absence of misfolded polypeptides in a sample. For example, ELISA, IHC, and/or RT-QuIC tests can be used to confirm the detection of the presence or absence of misfolded polypeptides in a sample.

This document also provides methods and materials for treating a mammal (e.g., a human) identified as having a proteinopathy as described herein (e.g., based, at least in part, on the presence of fibrils and/or polypeptide aggregates formed from misfolded polypeptides). For example, a mammal identified as having a proteinopathy based, at least in part, on the presence of fibrils formed from misfolded polypeptides in a sample obtained from the mammal can be administered one or more (e.g., one, two, three, four, five or more) agents that can be used to treat a proteinopathy. Examples of agents that can be used to treat a proteinopathy include, without limitation, agents (e.g., small molecules, oligonucleotides, peptides, and engineered immune cells) that can target the misfolded polypeptide, nanoparticle based delivery systems, and any combinations thereof. For example, a mammal identified as having a proteinopathy based, at least in part, on the presence of fibrils formed from misfolded polypeptides in a sample obtained from the mammal can be subjected to one or more (e.g., one, two, three, four, five or more) therapies that can be used to treat a proteinopathy. Examples of therapies that can be used to treat a proteinopathy include, without limitation, physical therapy, occupational therapy, speech therapy, and any combinations thereof.

The invention will be further described in the following examples, which do not limit the scope of the invention described in the claims.

### EXAMPLES

### Example 1: Nanoparticle-Based Diagnosis of Transmissible Spongiform Encephalopathies and Related Protein-Misfolding Disorders

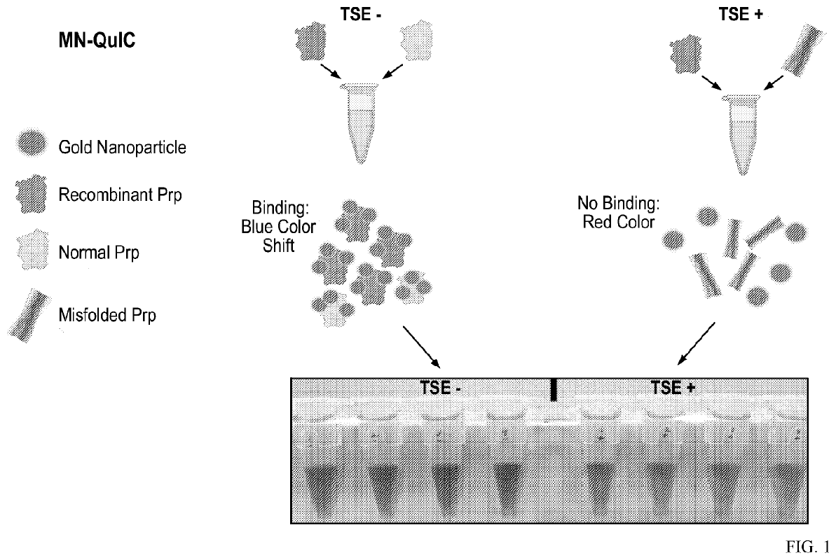

This Example describes the design of a method for detecting misfolded polypeptides that combines the incredible sensitivity of prion amplification methods with gold nanoparticle (AuNP)-based detection. The method described herein is also referred to as the Minnesota Quaking-induced Conversion (MN-QuIC) test (FIG. 1).

### Materials and Methods

### Experimental Design

Samples from 13 CWD detected (positive) and 13 CWD not-detected (negative) white-tailed deer lymphoid tissues were selected for independent EP-QuIC and MN-QuIC analyses (Table 1). White tailed deer lymphoid tissue was homogenized in PBS (10% w:v) using a BeadBug™ Homogenizer on max speed for 90 seconds. Tubes contained 1.5 mm zirconium beads. All positive and negative samples were selected based on independent ELISA, IHC, and/or RT-QuIC results.

TABLE 1
Samples.

| Sample | CWD | | | | |
|---|---|---|---|---|---|
| ID | Status | RT-QuIC | EP-QuIC | MN-QuIC | Tissue* |
| 01 (−) | − | x | | x | Parotid |
| 028 (+) | + | x | | x | Parotid |
| 737 | + | x | | x | Tonsil |
| 744 | + | x | | x | Tonsil |
| 761 | − | x | | x | Tonsil |
| 762 | − | x | | x | Tonsil |
| 782 (8) | − | x | x | x | Tonsil |
| 783 (9) | − | x | x | x | Tonsil |
| 784 (10) | − | x | x | x | Tonsil |
| 785 (11) | − | x | x | x | Tonsil |
| 786 (12) | − | x | x | x | Tonsil |
| 787 (13) | − | x | x | x | Tonsil |
| 788 (14) | − | x | x | x | Tonsil |
| 789 (15) | − | x | x | x | Tonsil |
| 790 (16) | − | x | x | x | Tonsil |
| 791 (17) | − | x | x | x | Tonsil |
| 920 (1) | + | x | x | x | Retro |
| 921 | + | x | | x | Retro |
| 922 (2) | + | x | x | x | Retro |
| 923 | + | x | | x | Retro |
| 924 | + | x | | x | Retro |
| 925 (3) | + | x | x | x | Retro |
| 926 (4) | + | x | x | x | Retro |
| 927 (5) | + | x | x | x | Retro |
| 928 (6) | + | x | x | x | Retro |
| 929 (7) | + | x | x | x | Retro |

*Retro, retropharyngeal lymph nodes.

### EP-QuIC

EP-QuIC was performed using a modified RT-QuIC master mix. Specifically, the addition of ThT was excluded to avoid potential interference with downstream AuNP absorbance readings. A BMG FLUOStar® Omega plate reader and a ThermoMixer (Eppendorf) were utilized to perform EP-QuIC analyses. Plate reader reactions were performed at 420 and shaken at 700 RPM, double orbital for 57 seconds and then rested for 83 seconds for 24 hours. Each sample was run with 4 replicates and controls with 6 replicates. Thermomixer reactions were performed at 480 for 24 hours with 600 RPM. Each sample had a minimum of 3 replicates. Replicates were pooled at the end of the reaction for subsequent ThT fluorescence analysis. To ensure fibrillation of the recombinant substrate, 100 μL were taken from the pooled fractions and added to 1 μL of 1 mM ThT, and fluorescence was measured in RFUs. These values were compared to a negative control to determine relative amounts of fibrillation.

### MN-QulC

AuNPs. 15 nm gold nanospheres were used for all MN-QuIC experiments reported herein, although other particle shapes (rod, cube, star, etc.) and materials (silver, copper, etc.) have utility for the method. Nanoparticles were buffer exchanged by centrifuging 533 μL of nanoparticle solution (12,000 rpm; 10 minutes), removing 490 μL of supernatant, and resuspending in 320 μL of low NaCl PBS buffer (10 mM Na₂HPO₄, 2.7 mM KCl, 1.8 mM KH₂PO₄, pH to 7.4 with HCl). In this solution, the nanoparticles are stable for weeks.

Prion amplification. Recombinant hamster PrP (HaPrP90-231) was produced and filtered. 10% tissue homogenates were diluted in 0.1% SDS/1X PBS solution. 1X PBS, 1 mM EDTA, 170 mM NaCl, and 0.1 mg/mL HaPrPrP were mixed prior to the addition of 98 μL into wells on a black 96-well plate with clear bottoms. 2 μL of diluted 10% tissue homogenates were added in each well before the plate was sealed and shaken on either a plate reader or ThermoMixer C equipped with SmartBlock plate and Thermotop (Eppendorf). Plate reader reactions were performed at 42, for 24 hours with 700 RPM, and Thermomixer reactions were performed at 48, for 24 hours with 600 RPM. Each sample had a minimum of 3 replicates. The resultant products were visualized with the addition of gold nanoparticles (prepared as described below). Absorbance and visual color were recorded.

### Visualization of Misfolded Proteins by Gold Nanoparticles

Visualization of post amplified material with nanoparticles was achieved by diluting individual wells to 50% in 1×PBS, 1 mM EDTA, 170 mM NaCl, and 1.2 mM sodium phosphate. 40 μL of these dilutions were then added to 360 μL of buffer exchanged AuNPs. This solution was then left to react for 30 minutes, although often the color differences between negative and positive samples became visible to the naked eye within the first two minutes. After the 30 minutes, photographs of the tubes were taken. Three replicates of 100 μl were taken from the 400 μl AuNP mixture and pipetted into three separate wells of a 96 well plate. The absorbance spectrum was then taken of each well at wavelengths 300-1000 nm.

### Statistical Analysis

Results from the EP-QuIC and MN-QuIC were grouped into either a positive or negative pool based on the known infection status from IHC, ELISA, and RT-QuIC data. A Mann-Whitney, non-parametric, two-tailed t-test was performed to determine statistical significance between the groups. Significance was determined using an alpha level of p<0.05.

### Results and Discussion

### Gold Nanoparticles Reflect Prion Misfolding Measured by ThT

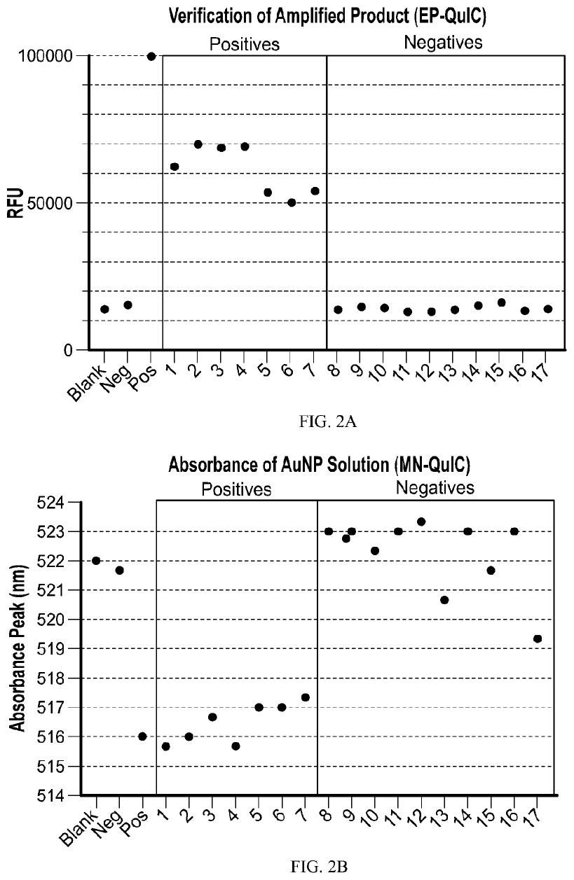

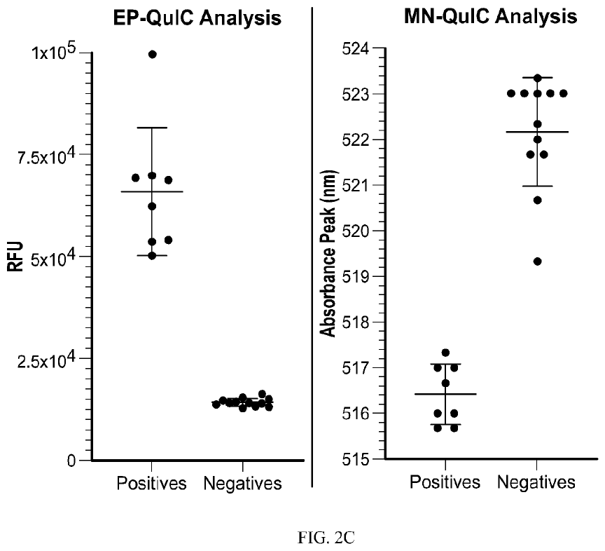

EP-QuIC analyses revealed successful prion amplification of CWD+ tissues (Table 1) as measured by ThT fluorescence versus negative controls. Relative fluorescence units of positive samples ranged from 50,122 RFU to 69,793 RFU, with the positive control at 99,639 RFU (FIG. 2A). CWD negative samples and controls ranged from 12,930 RFU to 16,208 RFU and with no evidence of prion amplification (FIG. 2A). In both EP-QuIC and MN-QuIC, the positive group was significantly different from the negative group (p<0.0001) (FIG. 2C). Independent MN-QuIC analyses of the same tissues used for EP-QuIC revealed unique absorbance values that clearly distinguished CWD+ samples from CWD− samples (FIG. 2B). Absorbance peaks for positive samples ranged from 515.5 nm to 517.5 nm, with the positive control at 516 nm. Alternatively, CWD− tissues revealed absorbance peaks ranging from 519.4 nm to 523.4 nm, including negative controls (FIG. 2B).

### Mechanism of Gold Nanoparticles for Distinguishing Normal Vs Misfolded Prions

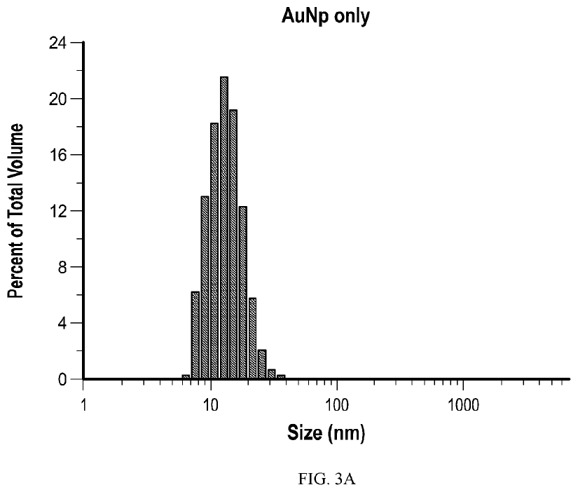

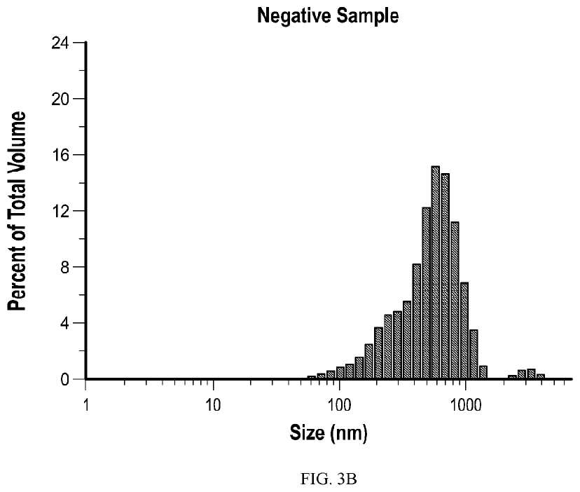

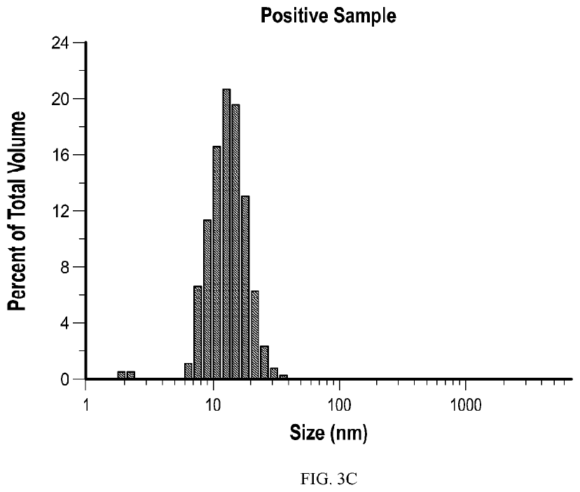

The optical properties of nanoparticles are extremely sensitive to their surrounding environment. While changes in the peak absorbed wavelength can be achieved by proteins simply absorbing onto the nanoparticles, very large peak absorbance shifts can come when nanoparticles aggregate together. These shifts due to aggregating can often be seen with the naked eye. After quaking and thermal treatment, if no fibrillation occurs in the prion substrate, prions added to AuNPs cause the AuNPs to aggregate compared to when no protein is present (FIGS. 3A and 3B). This aggregation causes a large peak absorbance shift. Thus, changing the color of the solution from red to blue. If after quaking and thermal treatment fibrillation occurs, there is a conformational change in the substrate prion. Prions in the fibril state do not cause AuNPs to aggregate (FIG. 3C). This means the nanoparticle solutions mixed with fibrillated proteins do not aggregate and thus the solution retains its red color.

### MN-QuIC is Comparable to RT-QuIC for Detecting Misfolded Prions

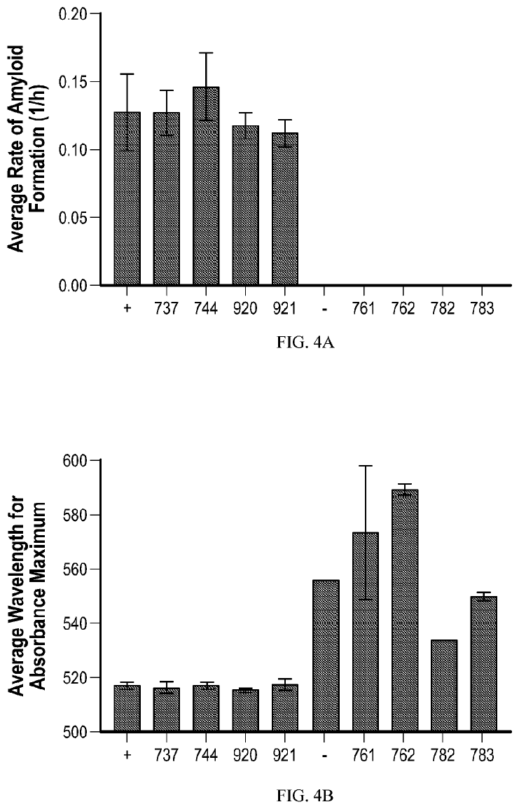

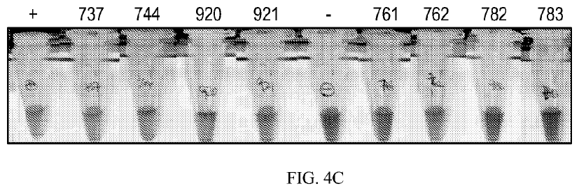

In some cases, MN-QuIC can be defined as the process where misfolded protein seeded amplification is performed using a combination of shaking and incubating with an appropriate substrate and results are immediately visualized using metallic nanoparticles. To show that MN-QuIC is comparable to RT-QuIC, the commonly used method for misfolded protein detection, 10% (w:v) lymphoid tissue homogenates from 5 positive and 5 negative animals were tested using both RT-QuIC and MN-QuIC. It was found that both absorbance of gold nanoparticles and visual color change from MN-QuIC accurately reflect results from standard RT-QuIC ThT reading (FIG. 4).

Together these results demonstrate that AuNP-based analyses of amplified prion products can rapidly (e.g., in less than 24 hours) detect misfolded polypeptides. This light-based assay for the detection of misfolded polypeptides is cost-effective and portable, and the results are readily distinguishable by eye or using a standard spectrophotometer.

### Example 2: Exemplary MN-QuIC Assay

### Methods

Amplify misfolded polypeptides

Shake sample for 24-48 hours.

Prepare metal nanoparticle solution

1A) Obtain 15 nm citrate capped AuNP in deionized (DI) water; concentration ˜ 2.7 nM.

2A) Aliquot 400 μL of AuNP solution into centrifuge tube.

3A) Spin at 12000 rpm for 10 minutes.

4A) Remove 360 μL leaving pelleted AuNP in 40 μL DI water.

5A) Add re-suspended pellet in 320 μL of low NaCl buffer.

Detect misfolded polypeptide

1B) Take sample directly from amplification, dilute to 25% protein v/v with End Point buffer.

2B) Add 40 μL 25% post amplification PrP solution to vial of 360 μL AuNP to bring the combined solution up to 400 μL.

3B) Let sit 30 minutes.

4B) Take picture of mixture and look for color change.

5B) Spin down mixture at 3000 rpm.

6B) Add 100 μL of mixture to three different wells on a 96-well plate.

7B). Insert plate into spectrometer and take the absorbance spectrum of each well.

### Results

### Color Shift

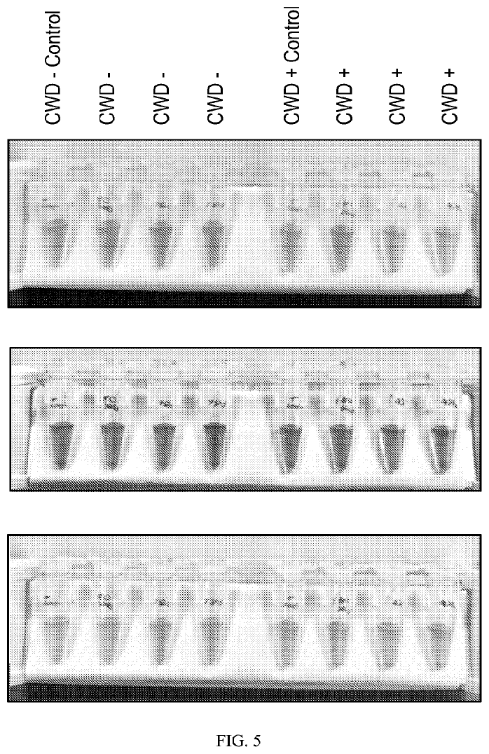

Pictures taken at step 4B are shown in FIG. 5. Each row was taken using a different camera filter to demonstrate that filters can be used to increase the contrast between positive and negative samples provided a positive and a negative control are present.

### Absorbance Shift

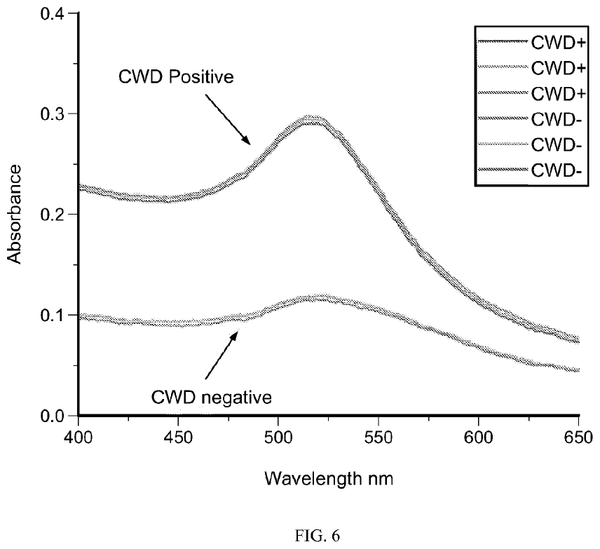

In addition to the color shift, a change in the absolute absorbance between positive and negative samples was seen because the AuNPs aggregate in the negative samples. The centrifugation step forces the larger/heavier aggregates out of solution and leaves a smaller absorption peak in the absorbance spectrum (FIG. 6).

### Peak Absorbance

The peak absorbance wavelength can also be used to determine the presence or absence of misfolded polypeptides.

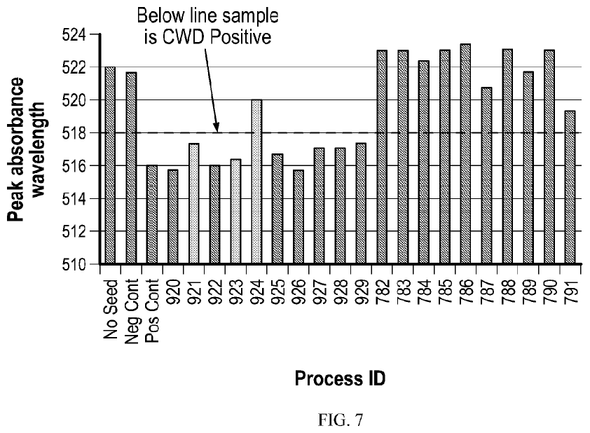

The absorbance spectrum was taken and the peak absorbance wavelength was found for each sample. CWD positive samples had peak absorbance wavelengths around 516 nm. Samples that were CWD negative had peak absorbance wavelengths around 521 nm (FIG. 7).

### Example 3: Exemplary MN-QuIC Assay for Tissue Samples

### 1) Purpose:

MN-QuIC is a method for detecting prionogenic fragments in a sample. It works by amplifying fibril formation through violent shaking and seeding with a sensitive form of prion, usually hamster PrP (HaPrP). The data is recorded in real-time.

### 2) Equipment Identifiers:

FLUOstar Omega plate reader

### 3) Materials:

MilliQ water
5X PBS buffer
N² media supplement (−20° C.)
0.1% SDS in PBS
10% (w:v) tissue homogenate (−80° C.)
HaPrP (90-231) [−80° C.]
100 kDa Pall MWCO filter
100 mM EDTA
2M NaCl
96-well plate

### 4) Procedure:

1. Fill out plate layout and all information included in plate layout file. Print this and use for reference as you set up the MN-QuIC
2. Save plate layout in MN-QuIC folder on desktop in a folder labeled with year, month, day_name Example:20200212_Suzanne
3. Put on Extra PPE (disposable lab coat and gloves, cut proof gloves if needed)
4. Take samples out of freezer and put in biosafety cabinet
5. Dissect tissues to subsample optimal tissue sample for testing on MN-QuIC (use cut proof gloves if needed)
6. Add 100 mg of tissue to labeled bead tube with 900 μL of PBS (use 1.5 mm Zirconium bead tubes)
7. Homogenize using BeadBeater at speed 400 for 120 seconds
8. Put samples in −80° C. until ready to run
9. Remove 5 tubes of HaPrP from −80ºC, Thaw, DO NOT VORTEX
10. Filter HaPrP with a 100 kDa spin column, 3,000 rpm, 15 minutes. Filter can be used twice. (Tubes are 500 μL aliquots, Max volume of filter is 500 μL) DO NOT VORTEX HaPrP
11. Carefully combine filtered HaPrP in a new tube and keep on ice. DO NOT VORTEX HaPrP
12. Determine concentration of filtered HaPrP using the Qubit (record results on plate layout second page)
   i) Standards: 10 μL standard+190 μL buffer
   ii) Samples: 1 μL sample+199 μL buffer
   iii) Vortex (including sample tube with HaPrP)
   iv) Incubate on benchtop for 15 minutes before reading
13. Determine the volume to add to a 100 μL rxn to get 0.1 mg/mL HaPrP.
   i) Formula: 0.01/concentration in mg/mL=mL of HaPrP/well
      1) Example: 0.0I/O.45 (mg/mL)=0.022 mL/well=22 μL/well
14. Prepare the rxn cocktail as follows, but do not add the HaPrP yet (scale up as needed, 1 full plate is ˜100 wells, refer to plate layout page 2):

| | | |
|---|---|---|
| | | Final |
| Component | Volume/well | Concentration |

| 5X PBS | 20 | uL | 1X | |
|---|---|---|---|---|
| 100 mM EDTA | 1 | uL | 500 | uM |
| 2M NaCl | 8.5 | uL | 300 | mM |
| HaPrP | 10 | ug | 0.1 | mg/mL |

| | | |
|---|---|---|
| H₂O | Up to 100 uL | N/A |

15. Vortex the cocktail without the HaPrP.
16. Before adding the HaPrP, filter the master mix through a 0.22 μm syringe filter.
17. Gently add the HaPrP and mix by inversion. DO NOT VORTEX
18. Gently transfer 98 μL to each well.
19. Dilute 100X 1:100 N2 media in 0.1% SDS in PBS.
   i) For full plate 50 μl of N2 media in 4.95 mL of 0.1% SDS in PBS
   ii) Add appropriate amount to each dilution tube
20. Make 1:10 serial dilutions of tissue homogenate in the prepared solution (N2 media diluted in 0.1% SDS in PBS) down to 10-10 (or required dilution). The original homogenate is considered 10-1.
   i) For 4 replicates of controls: Add 2 μL tissue homogenate to 18 μL prepared solution (10-2), securely close tube and vortex. Then take 2 μL of 10-2 dilution and add to 18 μL of prepared solution to make 10-3 dilution. Securely close tube and vortex. Continue this process until all dilutions are done
   ii) For up to 100 replicates of samples: Add 2 μL tissue homogenate to 198 μL prepared solution (10-3), securely close tube and vortex.
21. Gently add 2 μL of the sample and control tissue dilutions to each well.
22. Apply the transparent film to the plate to prevent evaporation, ensure wells are sealed tightly
23. Run the MN-QuIC script.
   i) Push button on front of plate reader to eject plate carriage
   ii) Place plate in plate carriage with A1 in the upper left hand corner
   iii) Remove gloves and dispose of in yellow barrel
   iv) Open the Omega software
   v) Login with username and password
   vi) Unplug the Internet cable from the back of the computer
   vii) Open Script Mode window
   viii) Change ID to date of run_name
      1) (year, month, day_name Example:20200212_Suzanne)
   ix) Click on Temp to turn on (set to 42° C. if not set)
      1) Click to turn incubator on
   x) Double check settings of script
      1) Temp 42° C.
      2) Loop 57
      3) Every 45 minutes
   xi) Click on Manage Protocol then Rocky mt shake 2 to double check conditions
      1) Layout only has A1 selected
      2) Shake 700 rpm, double orbital every 57 seconds
   xii) Click on Rocky Mtn Read to double check conditions
      1) Excite 450-10
      2) Emit 480-10
      3) Gain 1600
      4) Bottom Optic
      5) Set up plate layout (refer to your layout from step 1)
      6) No shaking
   xiii) Save Script
   xiv) Press Start
   xv) (may have to wait until reader reaches 42° C.) Press yes to begin read

### Example 4. A Field-Deployable Diagnostic Assay for the Visual Detection of Misfolded Prions

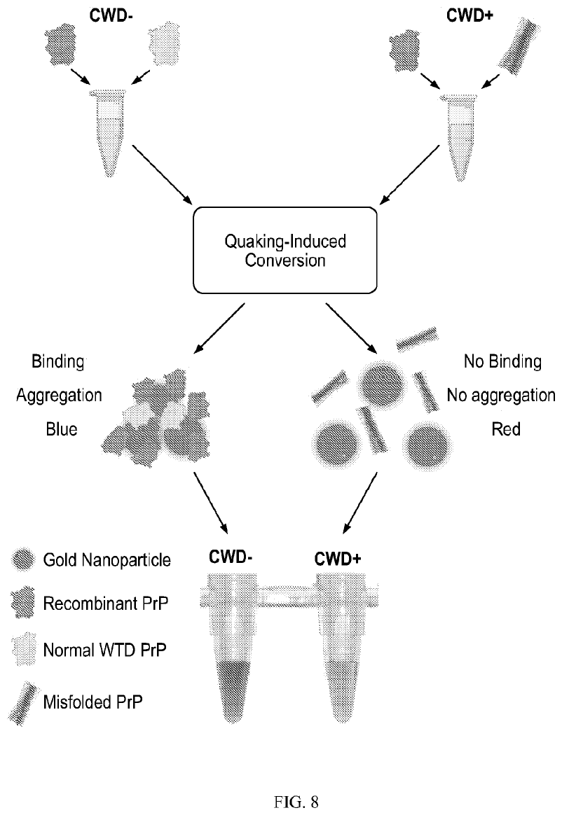

This Example describes the design of a nanoparticle-based assay that combines the unique color properties of AuNPs and the methods of quaking-based prion protein fibril amplification to detect the presence or absence of PrPCWD using both visual and spectroscopic methods (FIG. 8).

The results in this Example re-present and expand on at least some of the results provided in other Examples.

### Results

Comparison of Gold Nanoparticle Interaction with Recombinant Cellular Prions Vs Amplified Misfolded Fibrils

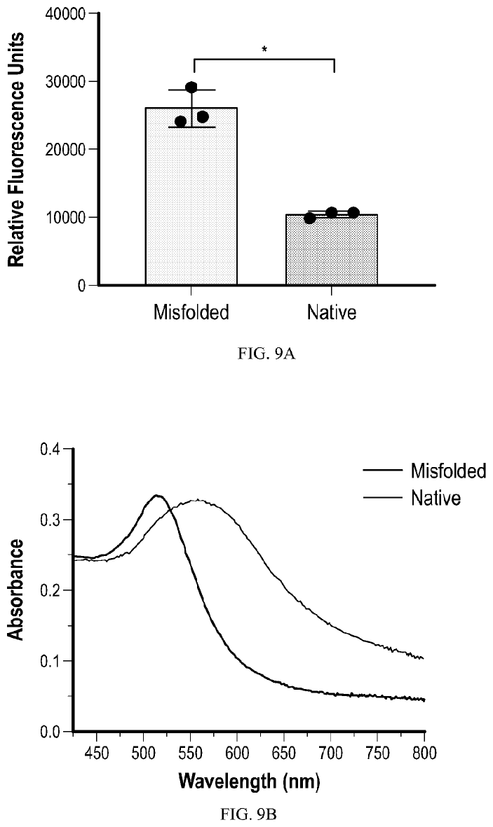

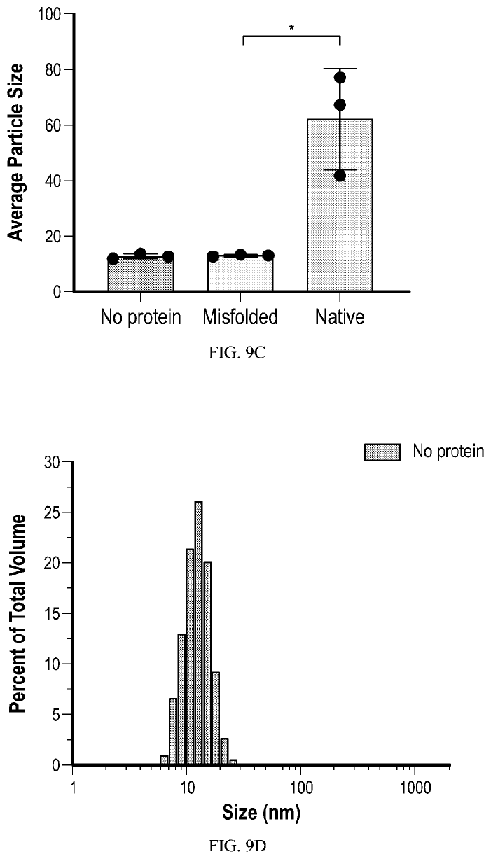

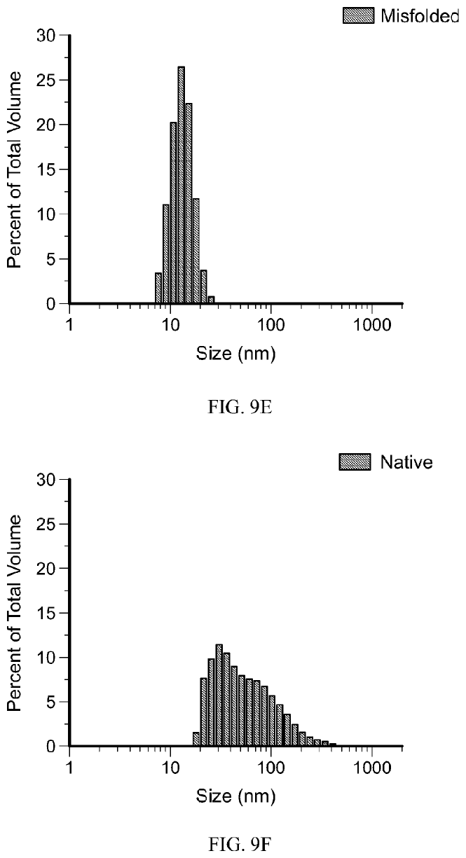

To investigate whether AuNPs can differentiate between misfolded fibrils from recombinant hamster prion protein (rHaPrP), two sets of reaction mixtures seeded with and without spontaneously misfolded rHaPrP prion fibrils were processed following modified RT-QuIC protocols without ThT. The presence of fibril formation was examined in all reaction mixtures by adding and quantifying ThT post hoc (FIG. 9A). ThT fluorescence was significantly different between misfolded rHaPrP (seeded) and native non-misfolded rHaPrP (no seed) (p-0.05; FIG. 9A). It was examined whether misfolded prion fibrils would interact differently with citrate capped AuNPs, as compared with native rHaPrP, and whether the interaction would influence AuNP aggregation as measured by dynamic light scattering (DLS). Following ThT quantification, misfolded rHaPrP samples and native rHaPrP samples were spiked into separate AuNP solutions. After a brief incubation period, there was a visible color difference between the AuNP solutions spiked with misfolded and native rHaPrP similar to the change in FIG. 8. This was also reflected in the visible spectrum of the absorbance peak (FIG. 9B). Color changes due to aggregation have been reported in literature for a variety of nanoparticle/protein combinations including prions and AuNPs. To quantify this, DLS experiments were performed on the three AuNP solutions and average effective particle size was determined for each sample (FIG. 9C). A significant difference of AuNP effective particle sizes was observed between AuNP solutions spiked with misfolded rHaPrP versus native rHaPrP (p=0.05) (FIG. 9C). The AuNPs spiked with no protein (blank) and misfolded rHaPrP exhibited similar particle size distributions (FIGS. 9D and 9E), indicating that the misfolded rHaPrP solutions did not induce aggregation. On the contrary, the addition of diluted native rHaPrP resulted in larger effective sizes for AuNPs (FIG. 9F) than AuNPs with no protein added (FIG. 9D), indicating that the addition of diluted native rHaPrP caused AuNPs to aggregate. These results indicate differential AuNP binding capacity between native rHaPrP and misfolded rHaPrP fibrils.

### CWD Positive and Negative Samples Produce Unique AuNP Optical Signatures

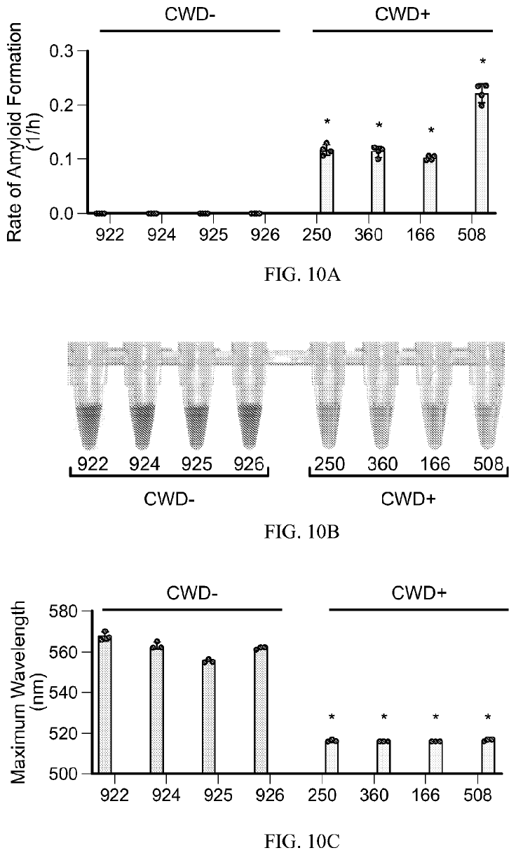

Understanding that pathogenic prions (PrpSc) can induce rHaPrP misfolding and amplification and thus influence AuNP aggregation (see above), the potential of MN-QuIC for CWD diagnostics using PrpCWD positive and negative white-tailed deer lymphoid tissues was investigated. Homogenates of independently confirmed CWD positive and negative white-tailed deer medial retropharyngeal lymph nodes (RPLN) were used (Table 2). Independent RT-QuIC analyses were performed on all tissues used for AuNP analyses (FIG. 10A). Thermomixers have been used previously in conjunction with end-point ThT readings (e.g., EP-QuIC) to determine the presence of CJD prion seeding activity. In light of the previous results, it was anticipated that AuNPs could be utilized to facilitate direct visualization of QuIC-amplified misfolded rHaPrP solutions that were seeded with CWD positive tissue using a standard bench-top thermomixer. To test this, the same set of samples were added to RT-QuIC mastermix without ThT on 96-well plates, which were then subjected to shaking/incubation cycles on a thermomixer for 24 hours. The post-amplification solutions were then diluted to 50% and added to AuNP solutions. It was found that CWD positive and negative samples could be clearly distinguish simply through color difference appreciable by naked eye; the QuIC-amplified CWD positive and negative samples were red and purple, respectively (FIG. 10B). In order to quantify the observations and measure statistical differences, the absorbance spectrum of the AuNPs was measured from 400-800 nm using a 96-well plate reader. In the resulting absorbance spectrum, AuNP solutions combined with QuIC-amplified CWD positive samples had absorbance peaks near 516 nm (FIG. 10C), similar to the 515 nm absorbance peak of the AuNPs prior to the addition of protein solutions. However, the negative sample absorbance peaks were shifted to longer wavelengths of approximately 560 nm (FIG. 10C), confirming that the purple color of AuNP solutions from QuIC products originating from CWD negative tissue samples was consistent with the observed purple color of AuNP aggregates associated with native rHaPrp (FIGS. 9C and 9F). Accordingly, the peak AuNP absorbance wavelength of CWD negative samples are significantly larger (p<0.05) than CWD-positive samples (FIG. 10C).

TABLE 2
Metadata of Examined Animals. MNPRO = Minnesota
Center for Prion Research and Outreach. RPLN =
medial retropharyngeal lymph node.

| | | | | | Official | RT-QuIC |
|---|---|---|---|---|---|---|
| Animal | | | | | CWD Test | Results |
| MNPRO | | | | Back- | Results for | for |
| ID | Tissue | Age | Sex | ground | Animal | Tissue |
| 166 | RPLN | Adult | Female | Wild | Positive | Positive |
| 250 | RPLN | Adult | Male | Wild | Positive | Positive |
| 360 | RPLN | Adult | Female | Wild | Positive | Positive |
| 508 | RPLN | Adult | Male | Wild | Positive | Positive |
| 922 | RPLN | Adult | Female | Wild | Negative | Negative |
| 924 | RPLN | Adult | Female | Wild | Negative | Negative |
| 925 | RPLN | Fawn | Female | Wild | Negative | Negative |
| 926 | RPLN | Fawn | Female | Wild | Negative | Negative |

Field deployment and higher throughput protocols To show the potential for a portable, field-deployable diagnostic proof of concept experiments were performed in a rural field-station. Pooled retropharyngeal lymph nodes, parotid lymph nodes and palatine tonsils tissues from 13 animals collected from the surrounding wild deer population were tested. Three of these animals (blinded to the field team) were CWD positive as determined by regulatory ELISA and IHC testing of medial retropharyngeal lymph nodes. Using a blinded testing approach, MN-QuIC successfully detected, via red AuNP solutions, all three CWD positive animals. Additionally we successfully detected, with purple AuNP solutions, 8 out of the 10 CWD negative (i.e., not detected by ELISA) animals. The field test gave false positive signals for 2 of the 10 CWD ELISA not detected samples. However, the retropharyngeal lymph nodes, parotid lymph nodes and palatine tonsils were later retested individually (not pooled) for the two false positive animals. It was found that when all three tissues for each animal were tested individually they all gave negative (purple AuNP solution) results via the MN-QuIC assay. These preliminary experiments demonstrate the potential utility of MN-QuIC as a portable, field-deployable diagnostic tool for researchers and agencies.

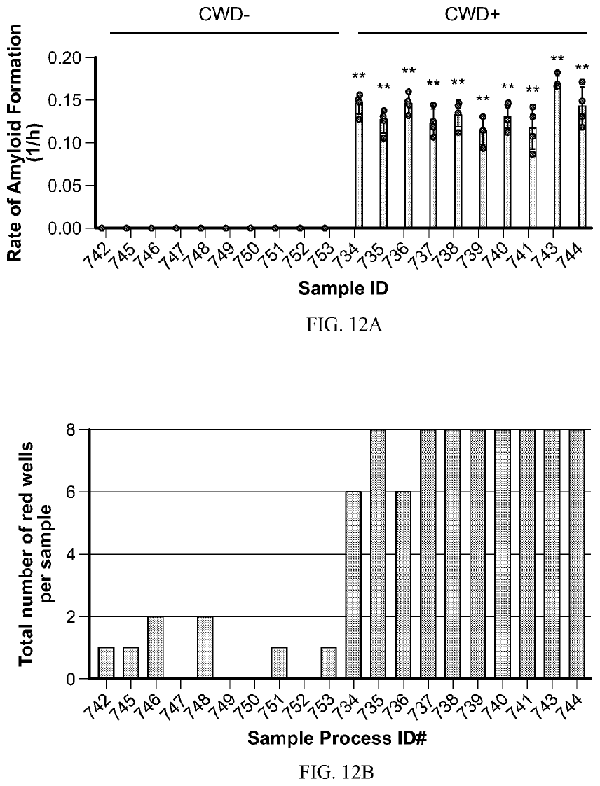

In order to demonstrate higher throughput protocols, palatine tonsil samples from a set of ten CWD negative and ten CWD positive white-tailed deer (Table 3) were tested. The status of these tissues was confirmed with RT-QuIC (FIG. 12A). For MN-QuIC, each sample had eight replicates and was prepared and subjected to the QuIC protocol in a 96-well plate on a thermomixer for 24 hours. A multichannel pipette was then used to add the QuIC amplified protein to a separate 96-well plate filled with AuNP solution. Color changes were observed within the first minute. For RT-QuIC analyses, it is common practice to consider a particular sample positive if 50% or more of its wells are positive. Using this approach, all 10 CWD positive animals were successfully identified using MN-QuIC (FIG. 12B). CWD negative samples were identified using a threshold of <50% of wells being red (i.e., majority purple in color) and 100% of CWD negative samples were correctly identified with the MN-QuIC assay (FIG. 12B). In addition to visual color, these results could also be measured by investigating the peak shift from the expected 517 nm. All red wells had absorbance peaks within 4 nm of 517 nm.

TABLE 3
Metadata of Examined Animals. MNPRO =
Minnesota Center for Prion Research and Outreach.

| | | | | | | Official | RT-QuIC |
|---|---|---|---|---|---|---|---|
| | Animal | | | | | CWD Test | Results |
| Process | MNPRO | | | | | Results for | for |
| ID | ID | Tissue | Age | Sex | Background | Animal | Tissue |
| 000734 | 000166 | Tonsil | Adult | Female | Wild | Positive | Positive |
| 000735 | 000197 | Tonsil | Yearling | Male | Wild | Positive | Positive |
| 000736 | 000250 | Tonsil | Adult | Male | Wild | Positive | Positive |
| 000737 | 000333 | Tonsil | Adult | Female | Wild | Positive | Positive |
| 000738 | 000360 | Tonsil | Adult | Female | Wild | Positive | Positive |
| 000739 | 000363 | Tonsil | Adult | Female | Wild | Positive | Positive |
| 000740 | 000376 | Tonsil | Adult | Female | Wild | Positive | Positive |
| 000741 | 000384 | Tonsil | Adult | Female | Wild | Positive | Positive |
| 000742 | 000498 | Tonsil | Yearling | Male | Wild | Negative | Negative |
| 000743 | 000508 | Tonsil | Adult | Male | Wild | Positive | Positive |
| 000744 | 000735 | Tonsil | Adult | Female | Wild | Positive | Positive |
| 000745 | 000065 | Tonsil | Adult | Female | Wild | Negative | Negative |
| 000746 | 000067 | Tonsil | Adult | Female | Wild | Negative | Negative |
| 000747 | 000091 | Tonsil | Adult | Female | Wild | Negative | Negative |
| 000748 | 000093 | Tonsil | Adult | Female | Wild | Negative | Negative |
| 000749 | 000230 | Tonsil | Fawn | Female | Wild | Negative | Negative |
| 000750 | 000232 | Tonsil | Adult | Female | Wild | Negative | Negative |
| 000751 | 000283 | Tonsil | Fawn | Male | Wild | Negative | Negative |
| 000752 | 000415 | Tonsil | Fawn | Female | Wild | Negative | Negative |
| 000753 | 000431 | Tonsil | Adult | Male | Wild | Negative | Negative |

### Electrostatic Forces Play a Role in AuNP CWD Detection

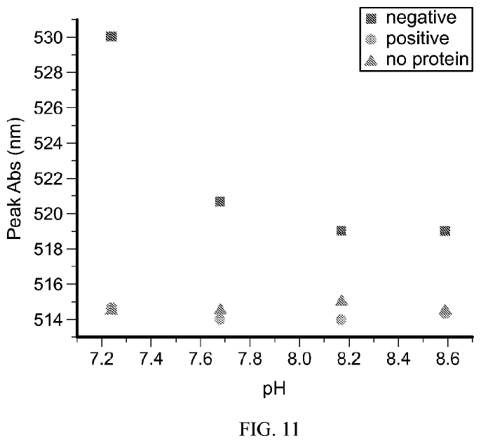

In light of the results described above, the potential mechanism underlying AuNP aggregation caused by rHaPrP solutions was examined. The theoretical isoelectric point (IP) of the rHaPrP is around pH 8.9 and because of this, rHaPrP is positively charged in the pH7.4 AuNP buffer, whereas citrate capped AuNPs are negatively charged even at pHs well below the buffer. Thus at pH 7.4, there exists an electrostatic attractive force between AuNPs and native rHaPrP that contributes to their interactions (aggregation and the color change). It was examined whether the charge on the protein would change when the pH of the environment is altered and that the interaction between AuNP and rHaPrP would be disrupted. As the pH of the AuNP solution was raised closer to the IP of rHaPrP, the absorbance peak shift from 515 nm of the AuNP-rHaPrP solution decreased (FIG. 11) while the control AuNP solution with no protein had very little peak deviation from 515 nm. This suggests that electrostatic interactions were at least partially responsible for facilitating the rHaPrP interactions with AuNPs. QuIC-amplified PrpCWD products, on the other hand, have experienced major conformational changes from its native form (as confirmed by ThT beta-sheet binding; FIG. 10) and have formed aggregates. This conformational change/aggregation may influence electrostatic attraction thus AuNP solutions with PrPCWD would remain around 515 nm (red) for all tested solutions (FIG. 11).

Together these results demonstrate that AuNP-based analyses of amplified prion products can rapidly (e.g., in less than 24 hours) detect misfolded polypeptides as a portable, sensitive field test.

### Methods

### Tissue Preparation

Twenty-eight white-tailed deer tissues (14 CWD-negative and 14 CWD-positive) were selected for RT-QuIC and MN-QuIC analyses. These samples were collected from white-tailed deer (Tables 2 and 3) and their CWD status was independently identified utilizing the Bio-Rad TeSeE Short Assay Protocol (SAP) Combo Kit (BioRad Laboratories Inc., Hercules, CA, USA). Positive RPLNs were confirmed by IHC. Metadata containing information of all specimens examined, including tissue type, are provided in Tables 2 and 3. White-tailed deer RPLNs and palatine tonsils were homogenized in PBS (10% w:v) in 2 mL tubes containing 1.5 mm zirconium beads with a BeadBug Homogenizer (Benchmark Scientific, Sayreville New Jersey, USA) on max speed for 90 seconds. All CWD positive and negative samples were selected based on independant ELISA, IHC, and/or RT-QuIC results and were subsampled using methods as described elsewhere (Schwabenlander et al., J. Wildl. Dis., doi: 10.7589/JWD-D-21-00033 (2021)).

### Preparation of Recombinant Substrate

Recombinant hamster PrP (HaPrP90-231) production and purification followed the methods described elsewhere (Schwabenlander et al., J. Wildl. Dis., doi: 10.7589/JWD-D-21-00033 (2021)). The substrate is derived from a truncated form (amino acids 90-231) of the Syrian hamster PRNP gene cloned into the pET41-a(+) expression vector and was expressed in Rosetta (DE3) E. coli.

### RT-QuIC for Cervid Lymph Tissues and Spontaneous Misfolding of Recombinant Prion Protein

For QuIC analysis, a master mix was made to the following specifications: 1X PBS, 1 mM ethylenediaminetetraacetic acid (EDTA), 170 mM NaCl, 10 μM thioflavin T (ThT), and 0.1 mg/mL rHaPrP. In instances where the end reaction would be analyzed using AuNPs, ThT could be excluded. The 10% tissue homogenates were further diluted 100-fold in 0.1% Sodium Dodecyl Sulfate (SDS) using methods described elsewhere (Schwabenlander et al., J. Wildl. Dis., doi: 10.7589/JWD-D-21-00033 (2021)) (final tissue dilution: 0.1%), and 2 μL of the diluent were added to each well containing 98 μL of RT-QuIC master mix. Spontaneous misfolding of recombinant prion protein was generated similarly but with unfiltered recombinant proteins and reagents. For these reactions, no infectious seed was necessary. The spontaneously misfolded material was used to seed reactions for the dynamic light scattering experiment, described below. Plates were amplified on a FLUOstar® Omega plate reader (BMG Labtech, Cary, North Carolina, USA; 42ºC, 700 rpm, double orbital, shake for 57 seconds, rest for 83 seconds). Fluorescent readings were taken at ˜45 minute increments.

### Thermomixer-based Amplification

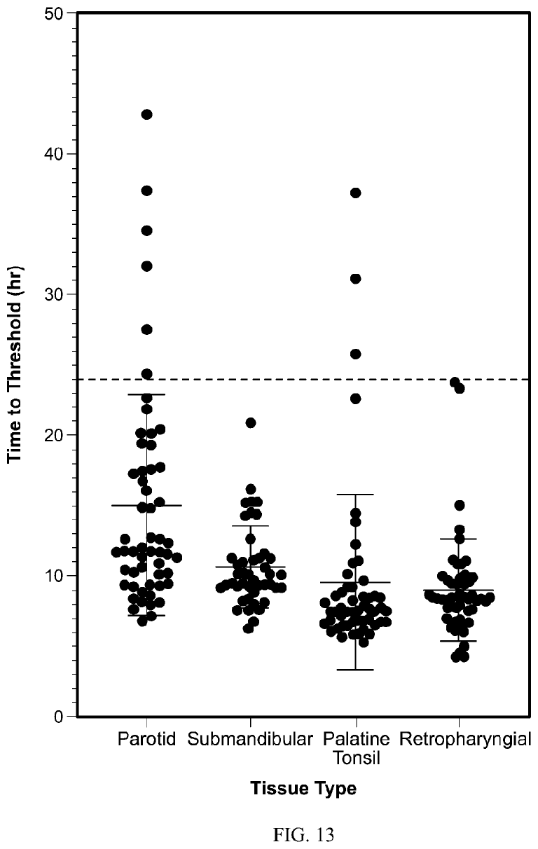

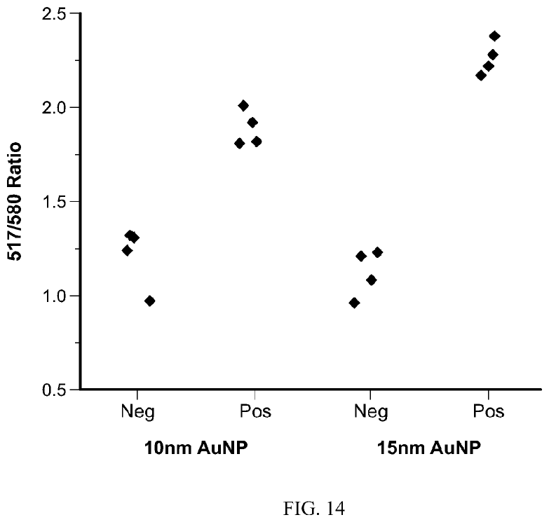

A standard benchtop shaking incubator (thermomixer) was leveraged to produce QuIC-based prion amplifications as described elsewhere (Cheng, et al., J. Clin. Microbiol. 54:1751-1754 (2016); and Vendramelli et al., J. Clin. Microbiol. 56:e00423-18 (2018)), although with slight modifications. Plates which were made for amplification on the thermomixer were prepared identical to those amplified on the plate reader. Reactions were performed on a ThermoMixerR C equipped with SmartBlock plate and Thermotop (Eppendorf, Enfield, Connecticut, USA) at 48ºC for 24 hours at 600 RPM (60s shake and 60s rest). A 24 hour run time was selected based on independent RT-QuIC results for medial retropharyngeal lymph nodes and palatine tonsils from CWD+ white-tailed deer reported as described elsewhere (Schwabenlander et al., J. Wildl. Dis., doi: 10.7589/JWD-D-21-00033 (2021)), including those examined herein, showing significant seeding activity within 9 to 24 hours (FIG. 13). The resultant products were visualized with the addition of gold nanoparticles (as described below).

### Preparation of Gold Nanoparticles (AuNP)

Post amplified material was visualized with 15 nm citrate capped gold nanoparticles purchased from usaNanopartz (Loveland, Colorado, USA) with stock concentrations ranging from 2.45 nM to 2.7 nM. AuNP protocols were modified from Springer et al. (Anal. Bioanal. Chem., 404:2869-2875 (2012)) and Zhang et al. (Talanta 89:401-406 (2012)). AuNPs were buffer exchanged using 530 μL of stock solution that was centrifuged in 1.6 mL tubes at 13,800 g for 10 minutes. 490 μL of supernatant was removed and the undisturbed pellet was resuspended with 320 μL of a low concentration phosphate buffer (PBSlow; PH 7.4 via addition of HCl) made of 10 mM Na₂HPO+(anhydrous), 2.7 mM KCl, 1.8 mM KH₂PO₄ (monobasic). After the quaking/incubation steps, protein solutions were diluted to 50% in MN-QuIC buffer, consisting of 1×PBS with the addition of final concentrations of 1 mM EDTA, 170 mM NaCl, 1.266 mM sodium phosphate. 40 μL of the protein diluted 50% in MN-QuIC buffer was then added to the 360 μL AuNP solution with ample mixing (results shown in FIGS. 10B and 10C). This solution was left to react at room temperature (RT) for 30 minutes (although a visible color change is observable within 60 seconds) before visual color was recorded (purple or red) and photographed. After images were taken, three replicates of 100 μL were taken from the 400 μL AuNP mixture and pipetted into three separate wells of a 96-well plate. The absorbance spectrum was then recorded for each well at wavelengths 400-800 nm using the FLUOstarR Omega plate reader (BMG Labtech, Cary, North Carolina, USA). For AuNP visualization experiments performed to determine higher throughput capacity (FIG. 12), proteins were prepared in the same way. After amplification on the thermomixer, proteins on a 96-well plate were diluted to 50% using MN-QuIC buffer and a multichannel pipette. 90 μL of AuNPs were then added to a separate 96-well plate. The AuNP wells were spiked with 10 μL of the diluted protein from the thermomixer (post-amplification) using a multichannel pipette. After waiting 30 minutes, the color changes were observed and the absorbance spectrum of the plate was taken.

### Field Deployment

The necessary MN-QuIC equipment was assembled as described above on two portable tables. Medial retropharyngeal lymph nodes, parotid lymph nodes and palatine tonsil were collected as described elsewhere (Schwabenlander et al., J. Wildl. Dis., doi: 10.7589/JWD-D-21-00033 (2021)), sampled and pooled together for each of the 13 animals tested. Tissues were subject to 24 hour MN-QuIC protocols as described above. Three replicates were performed for each of the 13 animals and, for field-based analyses, an animal was considered CWD positive if one or more replicates was red.

### Dynamic Light Scattering

Spontaneously misfolded rHaPrP samples (described above) were produced from solutions of rHaPrP with no seed added. A 96-well RT-QuIC reaction was performed with half the wells consisting of native rHaPrP seeded with spontaneously misfolded protein, and half consisting of native rHaPrP with no seed. The 96-well plate was then amplified using QuIC protocols described above. Post amplification, seeded samples were confirmed to have beta-sheet fibrillation while the non-seeded samples were confirmed to not have fibrillation based on ThT binding (described above). Seeded and non-seeded samples were diluted to 50% in MN-QuIC buffer and 40 μL of these solutions were added to 360 μL of AuNPs in PBSlow. Additionally, a blank with no protein was produced by adding 40 μL of MN-QuIC buffer to 360 μL of AuNPs in PBSlow. For native rHaPrP samples, color change could start to be observed within 1 minute of protein addition. No color change was observed in spontaneously misfolded rHaPrP samples at any time length. Dynamic light scattering measurements of all samples were taken after 5 minutes of protein addition using a Microtract NanoFlex Dynamic Light Scattering Particle Analyzer (Verder Scientific, Montgomeryville, PA, USA) and measurement times were 60 seconds. Five measurements were taken for each sample and then averaged.

### Effects of pH on the AuNP-Protein Interaction

In order to test the effects of pH on protein interacting with AuNP, four different 10 mM tris-buffer solutions with pHs ranging from 7.2 to 8.6 were created. Tris was used to give buffering for the desired pH range. AuNPs were buffer exchanged as described above except tris-buffer was used instead of PBSlow. Protein solutions were added as previously described.

### Additional Statistical Information

GraphPad Prism version 9.0 for Windows (GraphPad Software, San Diego, California USA, graphpad.com) was used for conducting statistical analysis. Three technical replicates were used to demonstrate the potential application of AuNP on spontaneously misfolded rHaPrP. For initial trials on RPLN tissues from eight (four positive and four negative) animals, four and three technical replicates used for RT-QuIC and AuNPs respectively. For plate-based protocols, palatine tonsils from ten positive and ten negative animals were tested using four and eight replicates for RT-QuIC and AuNPs, respectively. Unless specified in figures, rate of amyloid formation and maximum wavelength of samples were compared to negative controls on the respective plate. The one-tailed Mann-Whitney unpaired u-test (α=0.05) was used to test the average difference for all parameters of interests between samples.

### Example 5: Exemplary MN-QuIC Assay for Tissue Samples

### 1. Purpose

MN-QuIC is a method for detecting prionogenic fragments in a sample. It works by amplifying fibril formation through shaking and seeding with a sensitive form of prion such as HaPrP. Amplified proteins are then added to gold nanoparticles for detection.

### 2. Equipment

1. Centrifuge with 13.8 RFU capability
2. Optional: Spectrometer with 1 nm resolution absorbance measurement capability (for peak shift assay)
3. BeadBeater tissue homogenizer

### 3. Materials

1. PBS low NaCl (See appendix B for recipe)
2. Filtered Molecular Water
3. Filtered 5X PBS
4. Filtered 2M NaCL
5. Filtered 100 mM EDTA
6. 1 mM ThT (Made from Filtered Molecular Grade Water)
7. Filtered. 1% SDS PBS (appendix D)
8. 15 nm citrate capped AuNPs conc˜2.7 nM in DI water
9. 1.5 mL tubes
10. Post Amplified Sample material (from shaking protocol)
11. Black 96-well plate
12. Clear non-binding 96-well plate
13. Cuvettes (if using non plate reader spectrometer)
14. 1 mL pipette
15. 200 μL pipette
16. 1 mL pipette tips
17. 200 μL pipette tips
18. 96-tube racks (for holding samples)
   4. Tissue Prep (often done far in advance)
1. Take samples out of freezer and put in biosafety cabinet.
2. Dissect tissues for subsampling.
3. Add 100 mg of tissue to labeled bead tube with 900 μL 1X PBS (1.5 mm Zirconium bead tubes).
4. Homogenize using BeadBeater at max speed for 90 seconds.
5. Put samples in −80° C. until ready to run.

### 5. Amplification Procedure

1. Remove the appropriate amount of HaPrP tubes from −80° C. freezer.
2. Remove samples for testing from −80° C.
3. While the HaPrP is thawing, prepare the reaction master mix cocktail according to appendix A, but do not add the HaPrP yet. Note: 1.) Filter each solution with a 0.22 μm Syringe filter. 2.) The table gives the volumes for one 100 μL well on a 96-well plate. Scale up as many well as you need. 3.) HaPrP concentration varies between batches. Make the ratio of the water and HaPrP such that the final concentration of HaPrP in the 100 μL solution is 0.1 mg/mL.
4. Once thawed, filter the HaPrP with a 100 kDa spin column, 3,000×g, 15 minutes at 20° C.
5. When waiting for centrifuge to finish, make dilutions of tissues in 0.1% SDS PBS (Appendix D). The tissues should be in BeadBeater tubes (made in Tissue prep section) and should be considered to be 10-1 dilution already. Dilute tissues in 0.1% SDS PBS down to final dilution of 10-3 (so 100 times dilute the 10-1). Vortex each sample to make sure it's mixed.
6. Once HaPrP is done spinning down. Add the appropriate amount of HaPrP to the master mix (see Appendix A). DO NOT VORTEX. Gently invert a few times to mix.
7. Put 98 μL of master mix into each well of a black 96-well plate.
8. Add 2 μL of the dilutions made in step #5 to the wells.
9. Clear plate tape on the 96-well plate to stop evaporation
10. Put plate on thermo mixer with thermo top. Shaking: 600 RPM, Temp: 48° C., Time: 20 hours.

### 6. Nano-Particle Procedure

1. Pipette 1000 μL of AuNPs into a 1.5 μL centrifuge tube.
2. Spin down at 13.8 RFU for 15 minutes at room temperature.
3. Remove 960 μL of supranate. Be careful not to disturb the pellet!
4. Add 320 μL of PBS low NaCL (Appendix B). Repeat this for as many times as needed for desired volume of AuNP.
5. Remove 96-well plate with protein from thermo mixer.
6. Remove plate tape.
7. Add 90 μL of AuNPs in PBS low NaCL buffer to a clear, non-binding 96-well plate.
8. Add 10 μL of post amplified protein to each well.
9. Let sit 5 minutes. Color change between negative samples and positive samples should be apparent at this point.
10. Look for color change (can place against white background).
11. Optional: Look at absorbance spectrum in plate reader or cuvette based measurements. Look for changes in peak absorbance wavelength.
12. Dispose of all tubes, pipette tips and plates/cuvettes in yellow biohazard barrel.

### Appendix A

Filter each solution with a 0.22 μm Syringe filter.

The table gives the volumes for one well on a 96-well plate. Scale up as needed.

HaPrP concentration varies between batches. Make the ratio of the water and HaPrP such that the final concentration of HaPrP is 0.1 mg/mL.

### Master Mix

| | | |
|---|---|---|
| | | Final |
| Component | Volume/well | Concentration |

| 5X PBS | 20 | μL | 1X | |
|---|---|---|---|---|
| 100 mM EDTA | 1 | μL | 500 | μM |
| 1 mM ThT | 1 | μL | 50 | μM |
| 2M NaCl | 8.5 | μL | 300 | mM |
| HaPrP | 10 | μg | 0.1 | mg/mL |

| | | |
|---|---|---|
| H₂O | Up to 100 μL | N/A |

Appendix B: PBS low NaCl recipe
1.) Mix the following chemicals in DI water:

| | | |
|---|---|---|
| | | Weight for |
| | | Final molarity |
| Name | Final molarity | in 200 mL DI water |

| | | | | |
|---|---|---|---|---|
| Na₂HPO₄ (Anhydrous) | 10 | mM | 283 | mg |
| KCl | 2.7 | mM | 40 | mg |
| KH₂PO₄(Monobasic) | 1.8 | mM | 49 | mg |

2.) pH to 7.41 using HCl (about 4 drops of 3.7% HCl)

### Appendix C: 10 mM Sodium Phosphate

33.2 mL 1M Monobasic Sodium Phosphate
2.9 mL 1M Diabasic Sodium Phosphate
3564 mL H20
   Adjust pH to 5.8 with HCL/NaOH

### Appendix D: 0.1% SDS in PBS

1.) Mix 2370 μL Filtered Molecular Water, 600 μL 5X PBS, 30 μL 10% SDS
2.) Filter with 0.22 μL Syringe Filter

### Example 6. Analysis of MN-QuIC Assay Features

This Example describes the analysis of various parameters within a nanoparticle-based assay.

### Methods

A solution rich in native (non-misfolded) prions (PrP) was created.

CWD positive or CWD negative samples were seeded into a solution of native (non-misfolded) prions (PrP).

A MN-QuIC protocol was performed as described in Example 5 except that shaking was performed for 20 hours or 24 hours.

### Results

Different sized nanoparticles (10 nm and 15 nm) were used in a 24 hour MN-QuIC assay. For both 10 nm and 15 nm AuNPs, the samples containing CWD positive tissue appeared red and samples containing CWD negative tissue appeared blue. The colors of these samples are reflected in the peak absorbance spectra. The 517/580 ratio (e.g., the ratio of the absorbance at 517 nm divided by the absorbance at 580 nm) is shown in FIG. 14. These results demonstrate that different sized AuNPs can be used for a MN-QuIC assay.

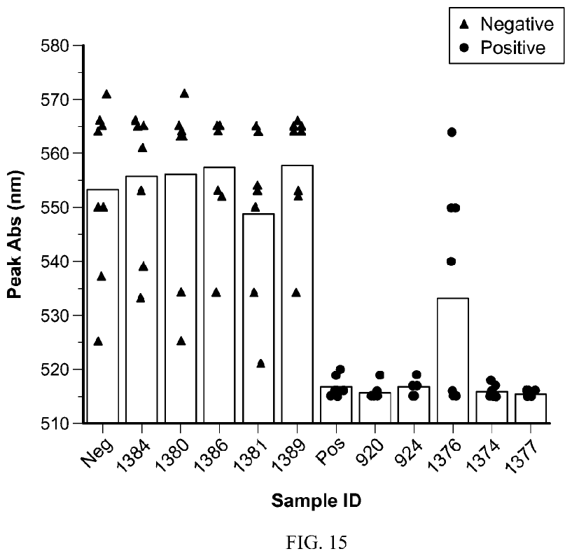

Different shaking times were used in another MN-QuIC assay. For this experiment, the shaking time was 20 hours. Samples containing CWD positive tissue appeared red, and samples containing CWD negative tissue appeared blue. The colors of these samples are reflected in their peak absorbance (FIG. 15). These results demonstrate that MN-QuIC assays can be done with different time scales.

### Example 7. Detection of Misfolded α-Synuclein Polypeptides with gold nanoparticles

This Example describes the use of a nanoparticle-based assay that combines the unique color properties of AuNPs and the methods of quaking-based prion protein fibril amplification to detect the presence or absence of misfolded α-synuclein polypeptides (e.g., to diagnose Parkinson's disease) using both visual and spectroscopic methods.

### Methods

A solution rich in native (non-misfolded) Recombinant hamster PrP (HaPrP) polypeptides was created.

Seeds of misfolded α-synuclein were put into a solution of native HaPrP as a positive sample (e.g., to mimic a proteinopathy associated with misfolded α-synuclein polypeptides such as Parkinson's disease). Seeds of native (non-misfolded) α-synuclein were also put into a solution of native (non-misfolded) HaPrP as a negative sample (mimicking a no disease).

A MN-QuIC protocol was performed largely as described in Example 5. After shaking, AuNP was applied to wells containing the positive and negative samples.

### Results

These experiments determine that misfolded α-synuclein polypeptides cross seed and induce misfolding in prion (PrP), thus allowing detection of misfolded α-synuclein polypeptides with an AuNP MN-QuIC assay.

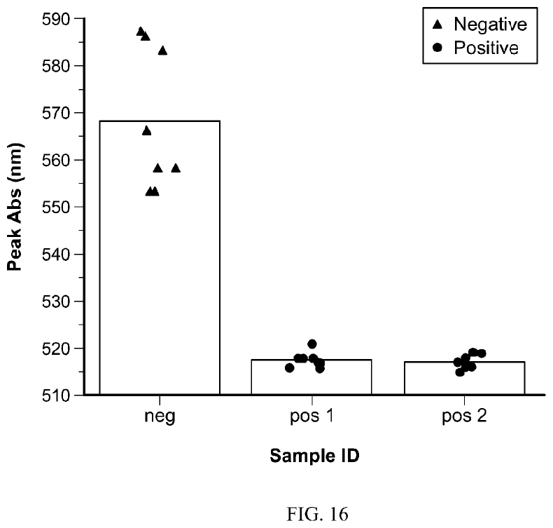

Samples were contacted with AuNPs. Samples containing misfolded α-synuclein polypeptides appeared red. Samples containing native α-synuclein polypeptides appeared blue. These colors were also reflected in the peak absorbance spectra (FIG. 16). These results demonstrate that a MN-QuIC assay can be used to detect misfolded polypeptides associated with human diseases.

### Example 8. Detection of Misfolded α-Synuclein Polypeptides via Organic Dye

This Example describes the use of an organic dye-based assay that combines the unique color properties of organic dyes (Congo Red) and the methods of quaking-based protein fibril amplification to detect the presence or absence of misfolded α-synuclein polypeptides (e.g., to diagnose Parkinson's disease) using both visual and spectroscopic methods.

### Methods

A solution rich in native (non-misfolded) α-synuclein polypeptides was created. Seeds of misfolded α-synuclein were put into a solution of native α-synuclein as a positive sample (e.g., to mimic a proteinopathy associated with misfolded α-synuclein polypeptides such as Parkinson's disease). Seeds of native (non-misfolded) α-synuclein were also put into a solution of native (non-misfolded) α-synuclein as a negative sample (mimicking a no disease).

A MN-QuIC protocol was performed similarly to the method as described in Example 5 (one notable difference is that α-synuclein was used as a substrate instead of PrP). After shaking, Congo Red was applied to wells containing the positive and negative samples. For samples contacted with Congo Red, samples were shaken on plate reader for 32 hours at 42° C.

### Results

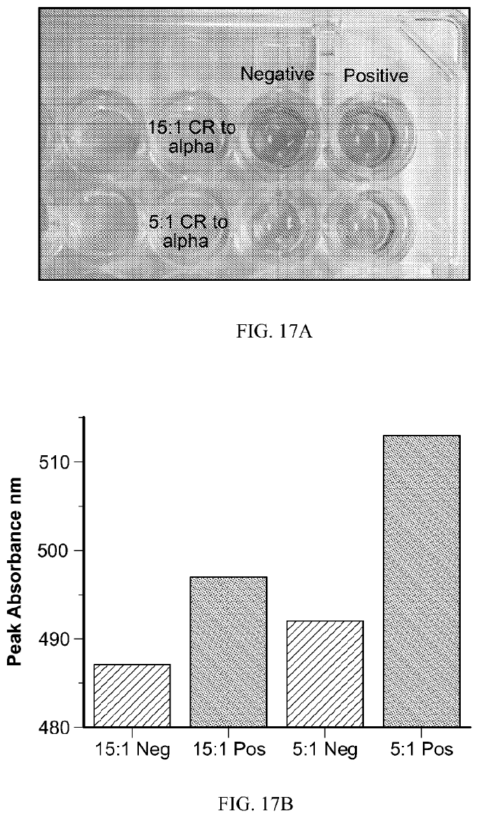

Samples were contacted with different ratios of Congo red to polypeptide (15:1 and 5:1 ratios). A color difference was visibly observed between the misfolded α-synuclein polypeptides and the native (non-misfolded) α-synuclein polypeptides. Samples containing misfolded α-synuclein polypeptides looked bluer then samples containing native (non-misfolded) α-synuclein polypeptides (FIG. 17A). This change is also reflected in the peak absorbance (FIG. 17B).

These results demonstrate that a MN-QuIC assay can be used to detect misfolded polypeptides associated with human diseases. These results also demonstrate that organic dyes (e.g., Congo Red) can be used in a MN-QuIC Assay (e.g., as an alternative to AuNPs).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting the presence or absence of misfolded polypeptides in a sample, said method comprising:
(a) amplifying said sample under conditions where said misfolded polypeptides, when present, form fibrils;
(b) contacting said sample with a solution containing metal nanoparticles;
(c) detecting said fibrils in said solution containing said metal nanoparticles;
(d) identifying said sample as having said presence of said misfolded polypeptides if said fibrils are detected; and
(e) identifying said sample as lacking said misfolded polypeptides if said fibrils are not detected.

2. A method for detecting the presence or absence of misfolded polypeptides in a sample, said method comprising:
(a) amplifying said sample under conditions where said misfolded polypeptides, when present, form globular polypeptide aggregates;
(b) contacting said sample with a solution containing metal nanoparticles;
(c) detecting said globular polypeptide aggregates in said solution containing said metal nanoparticles;
(d) identifying said sample as having said presence of said misfolded polypeptides if said globular polypeptide aggregates are detected; and
(e) identifying said sample as lacking said misfolded polypeptides if said globular polypeptide aggregates are not detected.

3. The method of any one of claims 1-2, wherein said sample is a biological sample.

4. The method of claim 3, wherein said biological sample is obtained from a living mammal.

5. The method of claim 4, wherein said living mammal is selected from the group consisting of humans, monkeys, camels, horses, mink, cats, cows, sheep, mice, rats, hamsters, brocket, chital, elk, fallow deer, marsh deer, mule deer, muntjac, moose, pampas deer, red deer, reindeer, roe deer, sambar deer, sika, white-tailed deer, antelope, and goats.

6. The method of claim 4 or claim 5, wherein said biological sample is selected from the group consisting of lymph tissue, muscle tissue, tonsil tissue, skin tissue, brain tissue, brain-stem tissue, blood, cerebrospinal fluid, urine, feces, saliva, mucus, liver tissue, heart tissue, intestinal tissue, spleen tissue, and eye tissue.

7. The method of claim 3, wherein said biological sample is obtained from a mammal post-mortem.

8. The method of claim 7, wherein said biological sample is beef or venison.

9. The method of any one of claims 1-2, wherein said sample is an environmental sample.

10. The method of claim 9, wherein said environmental sample is selected from soil, water, dust, and plants.

11. The method of claim 9 or claim 10, wherein said environmental sample is obtained using a swab or a filter.

12. The method of any one of claims 9-11, wherein said environmental sample is obtained from a location selected from group consisting of a natural habitat, a waterway, a farm, a food processing facility, a water-treatment facility, and a hospital.

13. The method of claim 12, wherein said environmental sample is obtained from said food processing facility, and wherein said food processing facility processes food intended for mammalian consumption.

14. The method of claim 12, wherein said environmental sample is obtained from said hospital.

15. The method of any one of claims 1-14, wherein said method comprises, prior to said amplifying step, isolating polypeptides from said sample.

16. The method of any one of claims 1-14, wherein said amplifying step comprises shaking said sample or sonicating said sample.

17. The method of any one of claims 1-16, wherein said metal nanoparticles are gold nanoparticles.

18. The method of any one of claims 1-16, wherein said method nanoparticles are selected from the group consisting of silver nanoparticles, copper nanoparticles, platinum nanoparticles, iron nanoparticles, and any alloys thereof.

19. The method of any one of claims 1-17, wherein said detecting step comprises visually detecting a color shift, wherein said color shift is indicative of the absence of said misfolded polypeptide.

20. The method of any one of claims 1-17, wherein said detecting step comprises detecting light absorbance, wherein an absorbance of from about 510 nm to about 521 nm is indicative of the presence of said misfolded polypeptide, and wherein an absorbance of from about 525 nm to about 600 nm is indicative of the absence of said misfolded polypeptide.

21. The method of any one of claims 1-20, wherein said misfolded polypeptide is selected from the group consisting of prion protein (PrP) polypeptides, tau polypeptides, amyloid ß polypeptides, α-synuclein polypeptides, and TDP-43 polypeptides.

22. The method of any one of claims 1-21, wherein said misfolded polypeptide is associated with a proteinopathy.

23. The method of claim 22, wherein said proteinopathy is selected from the group consisting of chronic wasting disease (CWD), Cruzefeldt-Jakob Disease, transmissible mink encephalopathy, feline spongiform encephalopathy, ungulate spongiform encephalopathy, bovine-spongiform encephalapothy, camilid spongiform encephalopathy, pituitary pars intermedia dysfunction (PPID), Alzheimer's Disease (AD), Parkinson's Disease (PD), Pick's disease, Lewy body dementia (LBD), amyotrophic lateral sclerosis (ALS), multiple systems atrophies, progressive supranuclear palsies, corticobasal degenerations, and chronic traumatic encephalopathies.

24. A method for detecting the presence or absence of misfolded polypeptides in a sample, wherein said method comprises:
(a) amplifying said sample under conditions where said misfolded polypeptides, when present, form fibrils;
(b) contacting said sample with a solution containing an organic dye;
(c) detecting said fibrils in said solution containing said organic dye;
(d) identifying said sample as having said presence of said misfolded polypeptides if said fibrils are detected; and
(e) identifying said sample as lacking said misfolded polypeptides if said fibrils are not detected.

25. A method for detecting the presence or absence of misfolded polypeptides in a sample, wherein said method comprises:
(a) amplifying said sample under conditions where said misfolded polypeptides, when present, form globular polypeptide aggregates;
(b) contacting said sample with a solution containing an organic dye;
(c) detecting said globular polypeptide aggregates in said solution containing said organic dye;
(d) identifying said sample as having said presence of said misfolded polypeptides if said globular polypeptide aggregates are detected; and
(e) identifying said sample as lacking said misfolded polypeptides if said globular polypeptide aggregates are not detected.

26. The method of any one of claims 24-25, wherein said sample is a biological sample.

27. The method of claim 26, wherein said biological sample is obtained from a living mammal.

28. The method of claim 27, wherein said living mammal is selected from the group consisting of humans, monkeys, camels, horses, mink, cats, cows, sheep, mice, rats, hamsters, brocket, chital, elk, fallow deer, marsh deer, mule deer, muntjac, moose, pampas deer, red deer, reindeer, roe deer, sambar deer, sika, white-tailed deer, antelope, and goats.

29. The method of claim 27 or claim 28, wherein said biological sample is selected from the group consisting of lymph tissue, muscle tissue, tonsil tissue, skin tissue, brain tissue, brain-stem tissue, blood, cerebrospinal fluid, urine, feces, saliva, mucus, liver tissue, heart tissue, intestinal tissue, spleen tissue, and eye tissue.

30. The method of any one of claims 24-25, wherein said biological sample is obtained from a mammal post-mortem.

31. The method of claim 30, wherein said biological sample is beef or venison.

32. The method of any one of claims 24-25, wherein said sample is an environmental sample.

33. The method of claim 32, wherein said environmental sample is selected from soil, water, dust, and plants.

34. The method of claim 32 or claim 33, wherein said environmental sample is obtained using a swab or a filter.

35. The method of any one of claims 32-34, wherein said environmental sample is obtained from a location selected from group consisting of a natural habitat, a waterway, a farm, a food processing facility, a water-treatment facility, and a hospital.

36. The method of claim 35, wherein said environmental sample is obtained from said food processing facility, and wherein said food processing facility processes food intended for mammalian consumption.

37. The method of claim 35, wherein said environmental sample is obtained from said hospital.

38. The method of any one of claims 24-37, wherein said method comprises, prior to said amplifying step, isolating polypeptides from said sample.

39. The method of any one of claims 24-37, wherein said amplifying step comprises shaking said sample or sonicating said sample.

40. The method of any one of claims 24-39, wherein said organic dye is selected from the group consisting of Congo Red, Nile Red, acridine orange, Trypan Blue, Evans Blue, Sirius Red f3b, primuline, X-34, 1,4-Bis(3-carboxy-4-hydroxyphenylethenyl)benzene, (trans, trans)-1-bromo-2,5-bis-(3-hydroxycarbonyl-4-hydroxy)styrylbenzene (BSB); BF-168, and (6-2-Fluoroethoxy)-2-[2-(4-methylaminophenil)ethenyl]benzoxazole.

41. The method of any one of claims 24-40, wherein said detecting step comprises visually detecting a color shift, wherein said color shift is indicative of the presence of said misfolded polypeptide.

42. The method of any one of claims 24-40, wherein said detecting step comprises detecting light absorbance, wherein an absorbance of from about 494 nm to about 550 nm is indicative of the presence of said misfolded polypeptide, and wherein an absorbance of from about 450 nm to about 493 nm is indicative of the absence of said misfolded polypeptide.

43. The method of any one of claims 24-42, wherein said misfolded polypeptide is selected from the group consisting of PrP polypeptides, tau polypeptides, amyloid ß polypeptides, α-synuclein polypeptides, and TDP-43 polypeptides.

44. The method of any one of claims 24-43, wherein said misfolded polypeptide is associated with a proteinopathy.

45. The method of claim 44, wherein said proteinopathy is selected from the group consisting of CWD, Cruzefeldt-Jakob Disease, transmissible mink encephalopathy, feline spongiform encephalopathy, ungulate spongiform encephalopathy, bovine-spongiform encephalapothy, camilid spongiform encephalopathy, PPID, AD, PD, Pick's disease, LBD, ALS, multiple systems atrophies, progressive supranuclear palsies, corticobasal degenerations, and chronic traumatic encephalopathies.

46. A method for detecting the presence or absence of misfolded polypeptides in a sample, said method comprising:
(a) amplifying said sample under conditions where said misfolded polypeptides, when present, form fibrils;
(b) contacting said sample with a solution containing quantum dots;
(c) detecting said fibrils in said solution containing said quantum dots;
(d) identifying said sample as having said presence of said misfolded polypeptides if said fibrils are detected; and
(e) identifying said sample as lacking said misfolded polypeptides if said fibrils are not detected.

47. A method for detecting the presence or absence of misfolded polypeptides in a sample, said method comprising:
(a) amplifying said sample under conditions where said misfolded polypeptides, when present, form globular polypeptide aggregates;
(b) contacting said sample with a solution containing quantum dots;
(c) detecting said globular polypeptide aggregates in said solution containing said quantum dots;
(d) identifying said sample as having said presence of said misfolded polypeptides if said globular polypeptide aggregates are detected; and
(e) identifying said sample as lacking said misfolded polypeptides if said globular polypeptide aggregates are not detected.

48. The method of any one of claims 46-47, wherein said sample is a biological sample.

49. The method of claim 48, wherein said biological sample is obtained from a living mammal.

50. The method of claim 49, wherein said living mammal is selected from the group consisting of humans, monkeys, camels, horses, mink, cats, cows, sheep, mice, rats, hamsters, brocket, chital, elk, fallow deer, marsh deer, mule deer, muntjac, moose, pampas deer, red deer, reindeer, roe deer, sambar deer, sika, white-tailed deer, antelope, and goats.

51. The method of claim 49 or claim 50, wherein said biological sample is selected from the group consisting of lymph tissue, muscle tissue, tonsil tissue, skin tissue, brain tissue, brain-stem tissue, blood, cerebrospinal fluid, urine, feces, saliva, mucus, liver tissue, heart tissue, intestinal tissue, spleen tissue, and eye tissue.

52. The method of claim 48, wherein said biological sample is obtained from a mammal post-mortem.

53. The method of claim 52, wherein said biological sample is beef or venison.

54. The method of any one of claims 46-47, wherein said sample is an environmental sample.

55. The method of claim 54, wherein said environmental sample is selected from soil, water, dust, and plants.

56. The method of claim 54 or claim 55, wherein said environmental sample is obtained using a swab or a filter.

57. The method of any one of claims 54-56, wherein said environmental sample is obtained from a location selected from group consisting of a natural habitat, a waterway, a farm, a food processing facility, a water-treatment facility, and a hospital.

58. The method of claim 57, wherein said environmental sample is obtained from said food processing facility, and wherein said food processing facility processes food intended for mammalian consumption.

59. The method of claim 58, wherein said environmental sample is obtained from said hospital.

60. The method of any one of claims 46-59, wherein said method comprises, prior to said amplifying step, isolating polypeptides from said sample.

61. The method of any one of claims 46-59, wherein said amplifying step comprises shaking said sample or sonicating said sample.

62. The method of any one of claims 46-61, wherein said quantum dots are selected from the group consisting of CdS, CdSe, CdTe, ZnS, ZnSe, PbS, and InP.

63. The method of any one of claims 46-62, wherein said detecting step comprises visually detecting a color shift, wherein said color shift is indicative of the absence of said misfolded polypeptide.

64. The method of any one of claims 46-63, wherein said detecting step comprises detecting light absorbance, wherein an absorbance of from about 510 nm to about 521 nm is indicative of the presence of said misfolded polypeptide, and wherein an absorbance of from about 525 nm to about 600 nm is indicative of the absence of said misfolded polypeptide.

65. The method of any one of claims 1-64, wherein said misfolded polypeptide is selected from the group consisting of PrP polypeptides, tau polypeptides, amyloid ß polypeptides, α-synuclein polypeptides, and TDP-43 polypeptides.

66. The method of any one of claims 1-65, wherein said misfolded polypeptide is associated with a proteinopathy.

67. The method of claim 66, wherein said proteinopathy is selected from the group consisting of CWD, Cruzefeldt-Jakob Disease, transmissible mink encephalopathy, feline spongiform encephalopathy, ungulate spongiform encephalopathy, bovine-spongiform encephalapothy, camilid spongiform encephalopathy, PPID, AD, PD, Pick's disease, LBD, ALS, multiple systems atrophies, progressive supranuclear palsies, corticobasal degenerations, and chronic traumatic encephalopathies.
